(19) United States
(12) Patent Application Publication
(10) Pub. No.: US 2005/0170369 A1
(43) Pub. Date: Aug. 4, 2005

(54) Methods for diagnosing a genetic risk for bone loss

(76) Inventors: Hitomi Shimpuku-Nosaka, Kobe (JP); Yasuhiro Nosaka, Kobe (JP); Kiyoshi Ohura, Nishinomiya (JP); Tatsuya Kawamura, Osaka (JP); Yoichi Tachi, Hachioji (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/898,558

(22) Filed: Jul. 26, 2004

(30) Foreign Application Priority Data
Feb. 2, 2004 (JP) 2004-25908

(51) Int. Cl. C12Q 1/68; G06F 19/00; G01N 33/48; G01N 33/50

(52) U.S. Cl. 435/6; 702/20

(57) ABSTRACT

The present invention provides means for diagnosing a genetic risk for bone loss after implant treatment. A genetic risk for bone loss after implant treatment is diagnosed by the method which comprises the following steps: (i) analyzing polymorphism at the base number position 9215 of the bone morphogenetic protein-4 gene in a nucleic acid sample; (ii) determining, based on information about polymorphism which was obtained in the step (i), the type of gene in the nucleic acid sample with respect to the polymorphism of the bone morphogenetic protein-4 gene; and (iii) diagnosing a genetic risk for bone loss based on the type of gene which was determined.

### CROSS REFERENCE TO RELATED APPLICATIONS

This application claims priority to Japanese application No. 2004-025908, filed on Feb. 2, 2004, whose content is hereby incorporated by reference in its entirety.

### FIELD OF THE INVENTION

This invention pertains to the utilization of the genes which are associated with bone loss after implant treatment. Concretely, it pertains to methods for detecting the types of the genes which are associated with bone loss after implant treatment, as well as methods for diagnosing a genetic risk for bone loss after implant treatment and kits for the methods, etc.

### BACKGROUND OF THE INVENTION

In recent years, preclinical or clinical research on the osseointegrated implant rendered it a highly predictable treatment, especially in the field of dentistry. However, the rate of success has not reached 100%, and bone loss around the implant has been directly responsible for failures (M. Esposito, J. M. Hirsch, U. Lekholm, and P Thomson. 1998. Biological factors contributing to failures of osseointegrated oral implants. (II). Etiopathogenesis. Eur J Oral Sci 106,3:721-764). To date, smoking history, brittle ossein, infection, excessive burden, and others have been reported as risk factors for bone loss (C. A. Bain and P. K. Moy. 1993. The association between the failure of dental implants and cigarette smoking. Int J Oral Maxillofac Implants 8,6:609-615; S. R. Bryant. 1988. The effects of age, jaw site, and bone condition on oral implant outcomes. Int J Prosthedont 11,5:470-490). In practical clinical settings, however, one experiences bone loss which is nonexplicable with these risk factors. Especially, early bone loss, observed at the time of second surgery in which no burden is applied to the implant, may develop even without infections or exposure of the cover screw (J. A. Toljanic, M. L. Banakis, L. A. Willes, and L. Graham. 1999. Soft tissue exposure of endosseous implants between stage I and stage II surgery as a potential indicator of early crestal bone loss. Int J Oral Maxillofac Implants 14, 3:436-441).

The inventors of the present invention have examined the relationship between the calcitonin receptor (hereafter also referred to as “CTR”) gene, which is involved in the formation and resorption of bone, and early bone loss and have reported that in the mandible the TC type of the CTR gene provokes early bone loss at a 20-fold odds ratio compared to the CC type (Y Nosaka, Y Tachi, H. Shimpuku, T. Kawamura, and K. Ohura. 2002. Association of calcitonin receptor gene polymorphism with early marginal bone loss around endosseous implants. Int J Oral Maxillofac Implants. 17:38-43). This result suggests that gene polymorphism of proteins which are involved in bone turnover possibly becomes a genetic risk factor for bone loss around the implant.

### SUMMARY OF THE INVENTION

Bone formation and remodeling occur actively around the implant during the healing period after first surgery, and many bone-metabolizing proteins are considered to be involved (W. E. Roberts. 1988. Bone tissue interface. J. Dent Educ 52, 12:804-809; L. P. Garetto, J. Chen, J. A. Parr, and W. E. Roberts. 1995; Remodeling dynamics of bone supporting rigidly fixed titanium implants; a histomorphometric comparison in four species including humans. Implant Dent 4, 4:235-243). Therefore, it appeared necessary to examine not only the CTR gene but also other genes. Bone morphogenetic protein-4 (hereafter also referred to as “BMP-4”), so far known as one of proteins that are involved in bone turnover, has been reported to present polymorphism within the human gene (M. Mangino, I. Torrente, A. De Luca, O. Sanchez, B. Dallapiccola, and G Novelli. 1999. A single-nucleotide polymorphism in the human bone morphogenetic protein-4 (BMP-4) gene. J. Human Genet 44, 1:76-77).

The present invention was made on the basis of the above background, and its objectives are to provide a means to assess in advance a genetic risk for bone loss after implant treatment and to contribute to the fulfillment of highly predictable implant treatment.

To accomplish the above objectives, the inventors of the present invention examined in patients who underwent implant treatment the relationship of early bone loss with given polymorphism of the CTR gene, with given polymorphism of the BMP-4 gene, and with the combination of both. Consequently, patients, who had a given genotype with respect to the CTR gene, were verified to prone to develop bone loss (namely, a high genetic risk for bone loss). Furthermore, patients, who had a given genotype with respect to the BMP-4 gene, were found to readily develop bone loss (namely, a genetic high risk for bone loss). There results provided the finding that the analysis of BMP-4 gene polymorphism is useful in assessing a genetic risk for bone loss. On the other hand, the combination of the results of the CTR gene polymorphism analysis with those of the BMP-4 gene polymorphism analysis was found to allow the assessment of a genetic risk for bone loss at a higher probability of prognosis.

The present invention was completed based on the above findings and provides the following compositions:
[1] A method for detecting the type of gene in a nucleic acid sample, comprising the following step:
   analyzing polymorphism at the base number position 9215 of the bone morphogenetic protein-4 gene in a nucleic acid sample.
[2] A method for detecting the type of gene in a nucleic acid sample, comprising the following steps (a) and (b):
   (a) analyzing polymorphism at the base number position 1377 of the calcitonin receptor gene in a nucleic acid sample; and
   (b) analyzing polymorphism at the base number position 9215 of the bone morphogenetic protein-4 gene in the nucleic acid sample.
[3] A method for diagnosing a genetic risk for bone loss after implant treatment, comprising the following steps (i) to (iii):
   (i) analyzing polymorphism at the base number position 9215 of the BMP-4 gene in a nucleic acid sample;
   (ii) determining, based on the information about polymorphism which was obtained in the step (i), the type of gene in the nucleic acid sample with respect to the polymorphism of the BMP-4 gene; and
   (iii) assessing, based on the type of the gene determined, a genetic risk for bone loss.
[4] The method for diagnosing a genetic risk according to [3], wherein:
   the presence or absence of allele A with respect to the polymorphism of the BMP-4 gene is determined in the step (ii); and
   in the step (iii), a genetic risk for bone loss is assessed to be high when determining in the step (ii) that allele A is present, and a genetic risk for bone loss is assessed to be low when determining in the step (ii) that allele A is absent.
[5] The method for diagnosing a genetic risk according to [3], wherein:
   the genotype with respect to the polymorphism of the bone morphogenetic protein-4 gene is determined either of the AV type, AA type, and VV type in the step (ii); and
   in the step (iii), a genetic risk for bone loss is assessed to be high when determining in the step (ii) that the genotype is the AV type or AA type, and a genetic risk for bone loss is assessed to be low when determining in the step (ii) that it is the VV type.
[6] A method for diagnosing a genetic risk for bone loss after implant treatment, comprising the following steps (I) to (V):
   (I) analyzing polymorphism at the base number position 1377 of the calcitonin receptor gene in a nucleic acid sample;
   (II) analyzing polymorphism at the base number position 9215 of the bone morphogenetic gene-4 in the nucleic acid sample;
   (III) determining, based on the information about polymorphism which was obtained by the step (I), the type of gene in the nucleic acid sample with respect to the polymorphism of the calcitonin receptor gene;
   (IV) determining, based on the information about polymorphism which was obtained by the step (II), the type of gene in the nucleic acid sample with respect to the polymorphism of the bone morphogenetic protein-4 gene; and
   (V) assessing a genetic risk for bone loss based on the types of the genes which were determined in the steps (III) and (IV).
[7] The method for diagnosing a genetic risk according to [6], wherein:
   the presence or absence of allele T with respect to the polymorphism of the CTR gene is determined in the step (III);
   the presence or absence of allele A with respect to the polymorphism of the BMD-4 gene is determined in the step (IV); and
   in the step (V), a genetic risk for bone loss is assessed to be high when determining the presence of allele T in the step (III) and/or when determining the presence of allele A in the step (IV), and a genetic risk for bone loss is assessed to be low in other cases.
[8] The method for diagnosing a genetic risk according to [6], wherein:
   the genotype with respect to the polymorphism of the calcitonin receptor gene is determined either of the TC type, TT type, and CC type in the step (III);
   the genotype with respect to the polymorphism of the bone morphogenetic protein-4 gene is determined either of the AV type, AA type, and VV type in the step (IV); and
   in the step (V), a genetic risk is assessed to be high when [the genotype of the calcitonin receptor gene which was determined in the step (III)/the genotype of the bone morphogenetic protein-4 gene which was determined in the step (IV)] is the TC type/AV type, TC type/AA type, TT type/AV type, IT type/AA type, CC type/AV type, CC type/AA type, TC type/VV type, or TT type/VV type, and a genetic risk is assessed to be low when it is the CC type/VV type.
[9] The method for diagnosing a genetic risk according to [6], wherein:
   the genotype with respect to the polymorphism of the calcium receptor gene is determined either of the TC type, TT type, and CC type in the step (III);
   the genotype with respect to the polymorphism of the bone morphogenetic protein-4 is determined either of the AV type, AA type, and VV type in the step (IV); and
   in the step (V), a genetic risk is assessed to be high when [the genotype of calcitonin receptor gene which was determined in the step (III)/the genotype of the bone morphogenetic protein-4 which was determined in the step (IV)] is the TC type/AV type, TC type/AA type, TT type/AV type, or TT type/AA type, and a genetic risk is assessed to be low when it is the CC type/AV type, CC type/AA type, TC type/VV type, TT type/VV type, or CC type/VV type.
[10] A kit for diagnosing a genetic risk for bone loss after implant treatment, comprising the following nucleic acid:
   a nucleic acid for analyzing polymorphism at the base number position 9215 of the bone morphogenetic protein-4 gene.
[11] A kit for diagnosing a genetic risk for bone loss after implant treatment, comprising the following (1) and (2):
   (1) a nucleic acid for analyzing polymorphism at the base number position 1377 of the calcitonin receptor gene; and
   (2) a nucleic acid for analyzing polymorphism at the base number position 9215 of the bone morphogenetic protein-4 gene.
[12] Fixing nucleic acids for diagnosing a genetic risk for bone loss after implant treatment, comprising the following nucleic acid which is fixed to an insoluble support:
   a nucleic acid for analyzing polymorphism at the base number position 9215 of the bone morphogenetic protein-4 gene.
[13] Fixing nucleic acids for diagnosing a genetic risk for bone loss after implant treatment, comprising the following (1) and (2) which are fixed to an insoluble support:
   (1) a nucleic acid for analyzing polymorphism at the base number position 1377 of the calcitonin receptor gene;
   (2) a nucleic acid for analyzing polymorphism at the base number position 9215 of the bone morphogenetic protein-4 gene.

In the methods of the present invention (methods for detecting the type of gene or methods for diagnosing), gene polymorphism which is associated with bone loss around the implant after implant treatment is analyzed. Then the type of gene in a nucleic acid sample with respect to the relevant gene polymorphism is detected based on the result of the above analysis. The use of information about polymorphism which is obtained by the detection of this type of gene allows the diagnosis of a genetic risk for bone loss. Thus, the present invention becomes a means which is efficacious in knowing in advance a genetic risk for bone loss when conducting implant treatment. Because of the provision of information which is effective in elucidating the mechanism by which bone loss develops after implant treatment, the present invention becomes also an important means in establishing methods to inhibit or prevent bone loss, etc.

### BRIEF DESCRIPTION OF THE DRAWINGS

These and other objectives and technical advantages of the present invention will be readily apparent from the following description of the preferred exemplary embodiments of the invention in conjunction with the accompanying drawings, in which:

FIG. 1 is a table which indicates the distribution of the BMP-4 genotype in the implant-placed patient group and the control group (Table 1).

FIG. 2 is a table which indicates the relationship between the implant placed and early bone loss (Table 2).

FIG. 3 is a table which indicates the distribution of risk factors in the bone loss group and the bone nonloss group (Table 3).

FIG. 4 is a table which indicates the results of a logistic analysis on risk factors for bone loss (Table 4).

FIG. 5 is a table which indicates the distribution of the CTR and BMP-4 genotypes in the implant treatment group and the healthy subject group (Table 5).

FIG. 6 is a table which indicates the distribution of known risk factors for bone loss in the bone loss group and the bon nonloss group (Table 6).

FIG. 7 is a table which indicates the distribution of the CTR and BMP-4 genotypes and the distribution of each allele in the bone loss group and the bone nonloss group (Table 7).

FIG. 8 indicates part of the sequence of the BMP-4 gene (complete cds).

FIG. 9 indicates part of the sequence of the BMP-4 gene (continuation of FIG. 8).

FIG. 10 indicates part of the sequence of the BMP-4 gene (continuation of FIG. 9).

FIG. 11 indicates part of the sequence of the BMP-4 gene (continuation of FIG. 10).

FIG. 12 indicates part of the sequence of the CTR gene (mRNA, complete cds).

FIG. 13 indicates part of the sequence of the CTR gene (continuation of FIG. 12).

### DETAILED DESCRIPTION OF THE INVENTION

The first aspect of the present invention is related to the methods for detecting the type of gene in a nucleic acid sample. One embodiment of the present invention is featured by the step of analyzing polymorphism at the base number position 9215 of the BMP-4 gene in a nucleic acid sample: 9215T→C [hereafter also referred to as “polymorphism BMP-4 (9215T→C)”]. On the other hand, other embodiment of the present invention is featured by the following steps (a) and (b):
(a) analyzing polymorphism at the base number position 1377 of the CTR gene in a nucleic acid sample: 1377T→C [hereafter also referred to as “polymorphism CTR (1377T C)”]; and
(b) analyzing polymorphism at the base number position 9215 of the BMP-4 gene in a nucleic acid sample [polymorphism (BMP-4 (9215T→C)].

Determination of the type of gene in a nucleic acid sample based on information about polymorphism which was obtained by conducting the above methods allows the definition of a genetic risk for bone loss. In the present specification, furthermore, “bone loss” means bone resorption around the implant after implant treatment, especially bone resorption which develops at the early stage after treatment. Furthermore, “a genetic risk for bone loss” means the degree of risk of developing bone loss mentioned above as determined by genetic factors. Moreover, “implant treatment” herein typically means the placement of an implant aiming at prosthodontic treatment in the field of dentistry but is used to cover a concept including also similar surgeries in fields other than dentistry.

In the above, descriptions such as 9215T→C means that polymorphism at the relevant base number position consists of two genotypes which are the bases before and after the arrow.

The base number and the polymorphism position of each gene in the present specification are expressed using as standards the known sequences which are registered in GenBank (http://www.ncbi.nlm.nih.gov/Genbank/index.html) and Molecular Databases (e.g., dbSNP: http://www.ncbi.nlm.nih.gov/SNP/), public databases which NCBI (http://www.ncbi.nlm.nih.gov/) offers.

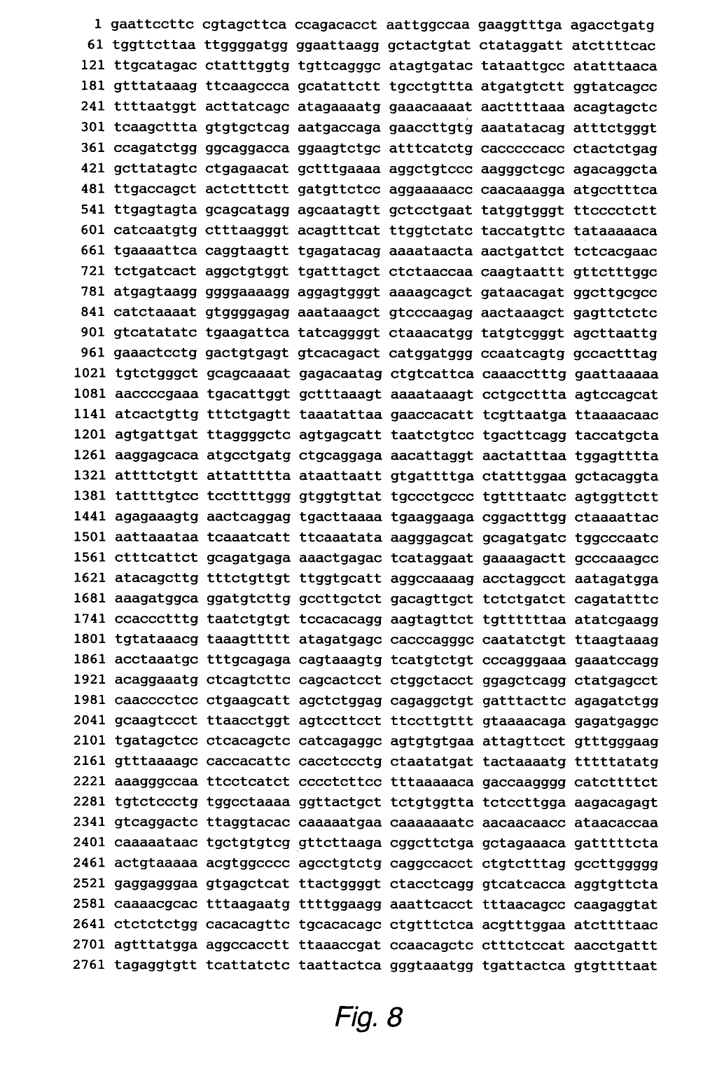

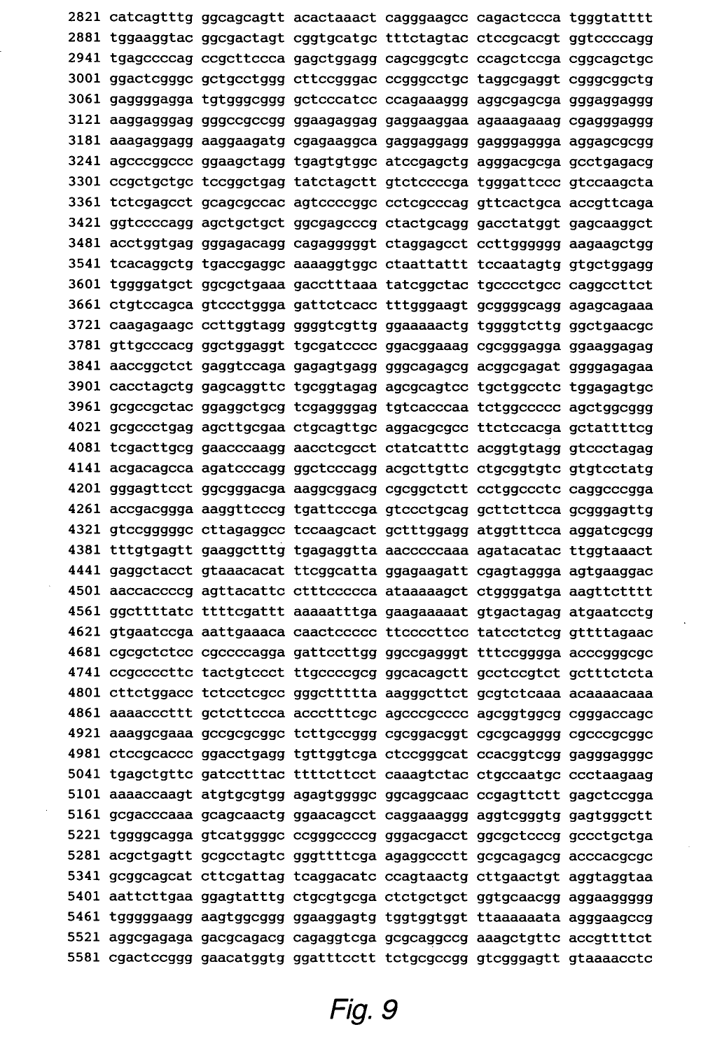

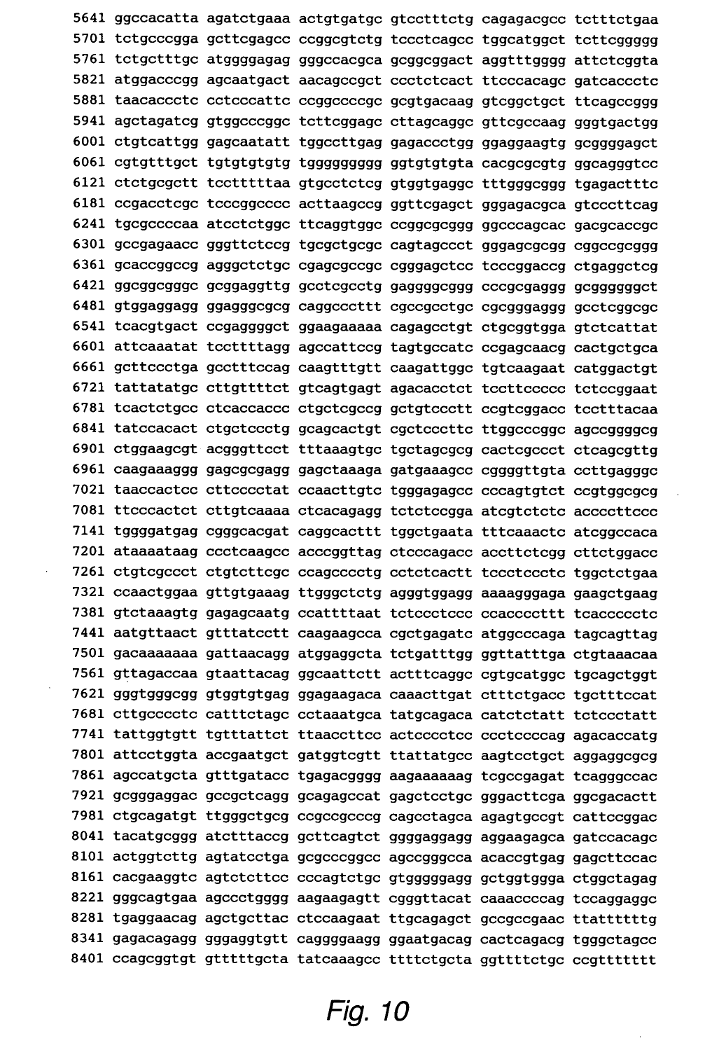

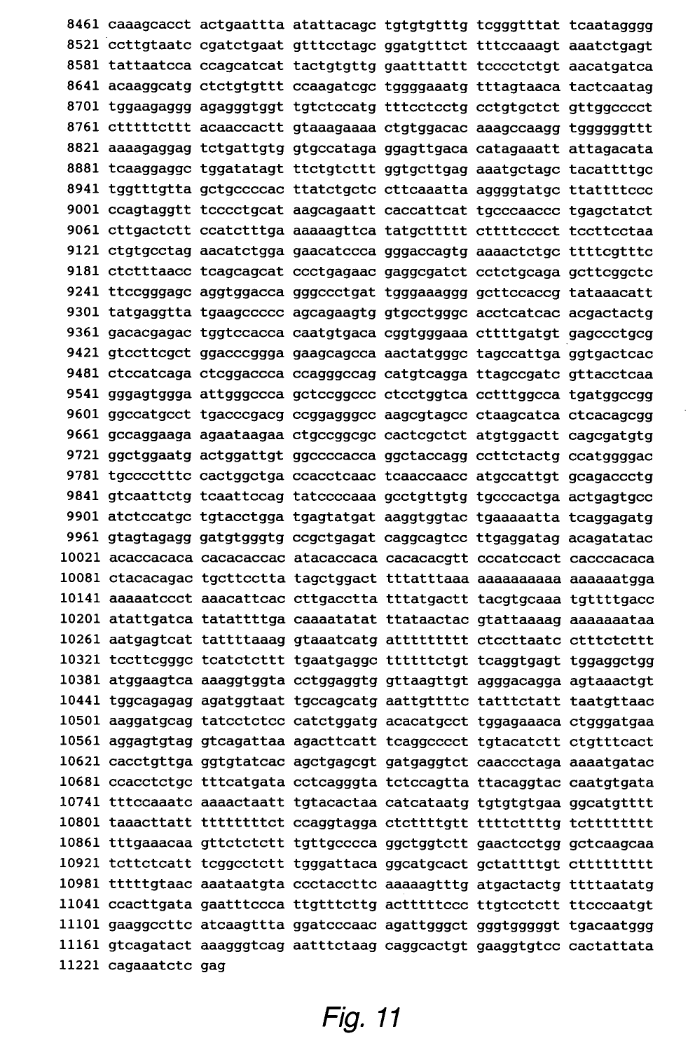

Polymorphism BMP-4 (9215T→C) is present in the exon 4 of the BMP-4 gene (genetic locus 14q21) (refSNP ID: rs17563 (http://www.ncbi.nlm.nih.gov/SNP/snp_ref.cgi?rs=17563; accession No. of mRNA including the relevant site of polymorphism: NM_{—}130851). In the base sequences in FIGS. 8 to 11 (SEQ ID NO: 1) (Genbank Accession No. U43842: Homo sapiens bone morphogenetic protein-4 (hBMP-4) gene, complete cds), furthermore, the 9215th base is the site of polymorphism BMP-4 (9215T→C).

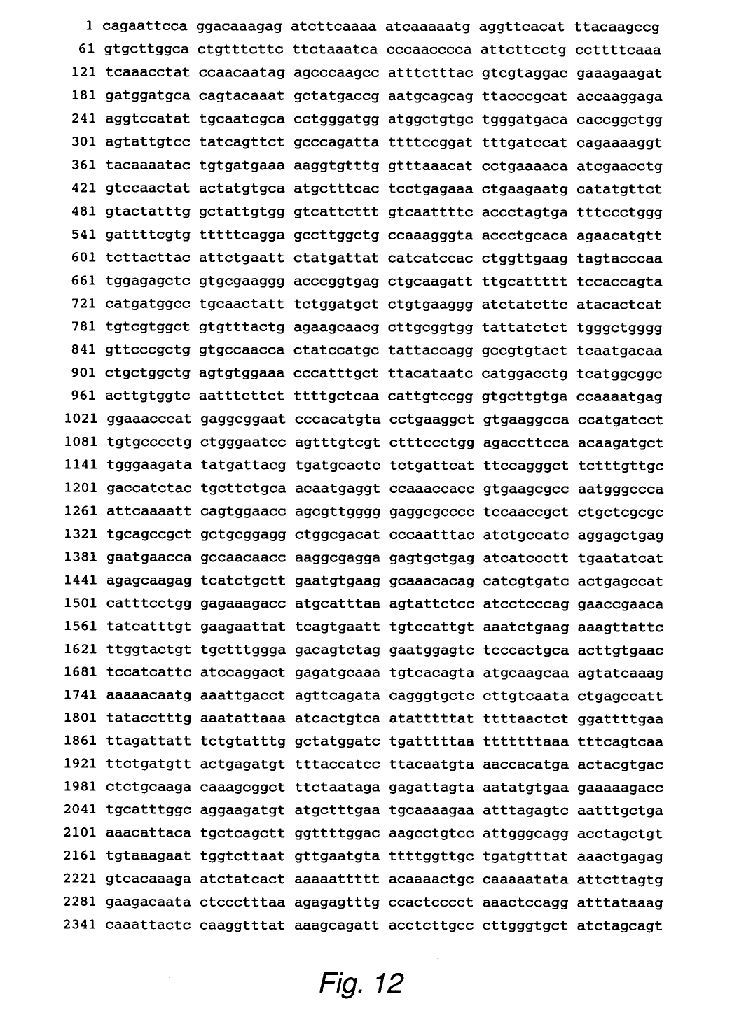

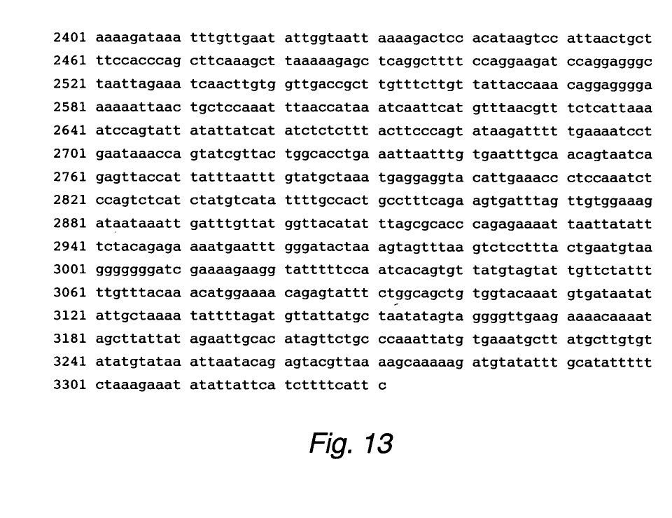

On the other hand, polymorphism CTR (1377T→C) is present in the 3 region of the CTR gene (genetic locus 7q21) (refSNP ID: 1801197) (http://www.ncbi.nlm.nih.gov/SNP/snp_ref.cgi?rs=1801197; Accession No. of mRNA including the relevant site of polymorphism: X69920). In the base sequences (Genbank Accession No. X69920: H. sapiens mRNA for calcitonin receptor) in FIGS. 12 to 13 (SEQ ID NO: 2), furthermore, the 1377th base corresponds to the site of polymorphism CTR (1377T→C).

In the present invention, “analyzing polymorphism” refers to the investigation on what type of gene in a nucleic acid sample has with respect to the site of polymorphism which is subject to the analysis and is synonymous to the investigation (determination) on the class of base (base sequence) at the position of the polymorphism. In the present invention, “type of gene” refers to a classification which is expressed by the presence or absence of a given allele, or a classification which is expressed as genotype that is a combination of alleles. Therefore, a step for “analyzing polymorphism” in the present invention investigates and determines the presence or absence of alleles of given polymorphism or the genotype (combination of alleles) in a nucleic acid sample. In the case that the above polymorphism of the BMP-4 gene is subject to the analysis, concretely, the presence or absence of allele A (or allele V), one of alleles with respect to the relevant polymorphism, or a nucleic acid sample is either of the AV type (heterojunction between allele A and allele V), AA type (homojunction between allele A and allele A), and VV type (homojunction between allele V and allele V) with respect to the BMP-4 gene is investigated and determined. Furthermore, “allele A” herein means an allele in which the base at the above site of polymorphism of the BMP-4 gene is C (cytosine) that renders the amino acid corresponding to the relevant site of polymorphism to Ala (alanine). Another “allele V” means an allele in which the base at the above site of polymorphism in the BMP-4 gene is T (thymine) that renders the amino acid at the corresponding site of polymorphism to Val (valine).

In the case that the above polymorphism of the CTR gene is subject to the analysis, on the other hand, a step for “analyzing polymorphism” in the present invention will investigate and determine the presence or absence of allele T (or allele C), one of alleles with respect to the relevant polymorphism, or that a nucleic acid sample is either of the TC type (heterojunction between allele T and allele C), TT type (homojunction between allele T and acyl T), and CC type (homojunction between allele C and allele C) with respect to the CTR gene. Furthermore, “allele T” herein means an allele in which the base at the above site of polymorphism of the CTR gene is T (thymine) that renders the amino acid corresponding to the relevant site of polymorphism to Leu (leucine). Another “allele C” means an allele in which the base at the above site of polymorphism in the CTR gene is C (cytosine) that renders the amino acid at the corresponding site of polymorphism to Pro (proline).

Methods for analyzing each genetic polymorphism are not limited in particular. Known methods may be employed, e.g., amplification by PCR using an allele-specific primer (and probe), a method to analyze polymorphism of amplified material by means of fluorescence or luminescence, PCR-RFLP (polymerase chain reaction-restriction fragment length polymorphism) method using PCR, PCR-SSCP (polymerase chain reaction-single strand conformation polymorphism) method [Orita, M. et al., Proc. Natl. Acad. Sci., U.S.A., 86, 2766-2770 (1989) et al.], PCR-SSO (polymerase chain reaction-specific sequence oligonucleotide) method, ASO [(allele specific oligonucleotide) hybridization method combining the PCR-SSO method and the dot hybridization method (Saiki, Nature, 324, 163-166 (1986) et al.], TaqMan-PCR [Livak, K J, Genet Anal, 14, 143 (1999), Morris, T. et al., J. Clin. Microbiol., 34, 2933 (1996)] method, Invader method [Lyamichev V et al., Nat Biotechnol., 17, 292 (1999)], MALDI-TOF-MS (matrix) method using the primer extension method [Haff L A, Smrnov I P, Genome Res 7, 378 (1997)], RCA (rolling cycle amplification) method [Lizardi P M et al., Nat Genet 19, 225 (1998)], a method using DNA microchip or microarray [Wang D G et al., Science 280, 1077 (1998) et al.], primer extension method, Southern blot hybridization method, dot hybridization method [Southern, E., J. Mol. Biol. 98, 503-517 (1975)]. Furthermore, an analysis may be made by direct sequencing of the portion of polymorphism which is subject to the analysis. Moreover, polymorphism may be analyzed by combining these methods ad libitum. In addition, any of the above analytical methods can be applied after amplifying in advance a nucleic acid sample (including the amplification of a region in a nucleic acid sample) according to nucleic acid-amplifying procedures including PCR and PCR amplification methods.

In the case of analyzing a number of nucleic acid samples, it is especially preferable to use analytical methods which allow the analysis of a number of samples in a relatively short period of time, e.g., allele-specific PCR method, allele-specific hybridization method, TaqMan-PCR method, Invader method, MALDI-TOF/MS (matrix) method using the primary extension method, RCA (rolling cycle amplification) method, and methods using the DNA chip or microarray.

The above methods use nucleic acids (also called “nucleic acids for polymorphism analysis” in the present invention), e.g., primer and probe in accordance with each method. As an example of nucleic acids for polymorphism analysis, a nucleic acid with a sequence which is complementary to a given region, including the corresponding site of polymorphism (region of partial DNA) in the gene which includes polymorphism that is subject to the analysis, may be mentioned. Furthermore, a nucleic acid, which has a sequence that is complementary to a given region, including the relevant site of polymorphism in the gene including polymorphism that is subject to the analysis (region of partial DNA) and which is designed to allow the specific amplification of the DNA fragment containing the relevant site of polymorphism (primer), may be mentioned. In the case that polymorphism at position 9215 of the BMP-4 gene, for example, a nucleic acid with a sequence that is complementary to the region of partial DNA including the base at position 9215 of the BMP-4 gene in which the base at position 9215 is T (thymine), or a nucleic acid with a sequence which is complementary to the region of partial DNA that contains the base at position 9215 of the BMP-4 gene in which the base at position 9215 is C (cytosine) corresponds to such a nucleic acid.

As other concrete examples of nucleic acids for polymorphism analysis, a nucleic acid set, which is designed to specifically amplify the region of partial DNA that contains the relevant site of polymorphism only in the case that the site of polymorphism subject to the analysis is either genotype, may be mentioned. More concretely, a nucleic acid set which is designed to specifically amplify the region of partial DNA including the site of polymorphism that is subject to the analysis and which consists of a sense primer that specifically hybridizes the region of partial DNA including the relevant site of polymorphism in the antisense chain whose site of polymorphism is either genotype and of an antisense primer that specifically hybridizes a partial region of the sense chain, can be mentioned. In the case that polymorphism at position 9215 of the BMP-4 gene is subject to the analysis, for example, a nucleic acid set, which is designed to specifically amplify the region of partial DNA containing the base at position 9215 of the BMP-4 gene and which consists of a sense primer that specifically hybridizes the region of partial DNA containing the base at position 9215 in the antisense chain of the BMP-4 gene whose base at position 9215 is T (thymine) and of an antisense primer that specifically hybridizes a region of the sense chain, or a nucleic acid set, which consists of a sense primer that specifically hybridizes the region of partial DNA including the base at position 9215 in the antisense chain of the BMP-4 gene whose base at position 9215 is C (cytosine) and of an antisense polymer that specifically hybridizes a partial region of the sense chain, corresponds to such a nucleic acid set. The length of the region of partial DNA to be amplified here is set accordingly in a range which is appropriate for its detection, for example, 50 to 200 bp, and preferably 80 to 150 bp.

The above nucleic acid primers and nucleic acid probes are mere examples. Nucleic acid primers may contain a partially modified base sequence in limits which allow the aimed amplification reaction without inconvenience, while nucleic acid probes may contain a partially modified base sequence in limits which allow aimed hybridization reaction without inconvenience. “Partial modification” herein means that part of bases is deleted, replaced, inserted, and/or added. The numbers of modified bases are one to seven for example, are one to five preferably, and are one to three more preferably. Furthermore, such a modification is made in the portions other than bases which correspond to the site of polymorphism, in principle.

As nucleic acids for polymorphism analysis (probes or primer), DNA fragments or RNA fragments are used accordingly in response to the analytical method employed. The base length of nucleic acids for polymorphism analysis may be sufficient if it exerts respective functions of each nucleic acid. Base lengths in the case of use as primers are 10 to 50 bp for example, are 15 to 40 bp preferably, and are 15 to 30 bp more preferably.

In the case of use as primers, some mismatches to the sequence which constitutes the template may be admitted as long as the primer can specifically hybridize the subject for amplification (template) and amplify the target DNA fragment. In the case of probes, some mismatches to the sequence which is subject to detection may be similarly admitted as long as the probe can specifically hybridize the sequence which is subject to detection. The numbers of mismatches are one to several, are one to five preferably, and are one to three more preferably.

Nucleic acids for polymorphism analysis (primers and probes) can be synthesized in accordance with known methods, e.g., phosphodiester method. Furthermore, textbooks (e.g., Molecular Cloning, Third Edition, Cold Spring Harbor Laboratory Press, New York) can be referred with respect to the design, synthesis, and others of nucleic acids for polymorphism analysis.

As shown in the Examples below, polymorphism BMP-4 (9215→C) and polymorphism CTR (1377T→C) can be readily analyzed according to the PCR-RFLP method. The primer for amplification to be used in the PCR-RFLP method can specifically amplify the DNA fragment including the site of polymorphism which is subject to the analysis, and no particular regard is given to its sequence, length, and others if it can provide a product of amplification whose length is appropriate for subsequent operations. One example of the primer set for amplification which can be used for the analysis of polymorphism BMP-4 (9215T→C) is shown below.

| | |
|---|---|
| Forward: | |
| 5′-GCTATCTCTTGACTCTTCCATC-3′ | (SEQ ID NO: 3) |
| Reverse: | |
| 5′-CATAGTTTGGCTGCTTCTCC-3′ | (SEQ ID NO: 4) |

Similarly, one example of the primer set for amplification which can be used for the analysis of polymorphism CTR (1377T→C) is shown below.

| | |
|---|---|
| Forward: | |
| 5′-TTCAGTGGAACCAGCGTTGG-3′ | (SEQ ID NO: 5) |
| Reverse: | |
| 5′-CTCAGTGATCACGATACTGT-3′ | (SEQ ID NO: 6) |

On the other hand, restriction enzymes for the PCR-RFLP method also are not particularly limited if they can provide products of digestion which reflect differences in the site of polymorphism which is subject to the analysis. For example, Hphl can be mentioned as an example of restriction enzymes which can be used for the analysis of polymorphism BMP-4 (9215T→C), and Alul as an example of restriction enzymes which can be used for the analysis of polymorphism CTR (1377T→C), respectively.

Nucleic acids for polymorphism analysis in the present invention can be labeled in advance with labeling substances. The use of such labeled nucleic acids allows, for example, the analysis of polymorphism by using the labeling amount in the product of amplification as a marker. Furthermore, labeling with reciprocally different labeling substances of two classes of primers which were designed to specifically amplify the region of partial DNA in the gene of each genotype that constitutes polymorphism allows discrimination of the genotype of a nucleic acid sample according to the labeling substance and labeling amount to be detected based on the product of amplification. As concrete examples of detection methods using these labeled primers, a method to detect polymorphism can be mentioned, which labels with fluorescein isocyanate and Texas red two classes of nucleic acid primers that respectively and specifically hybridize the sense chain of each genotype constituting polymorphism (allele-specific sense primers), which amplifies the region of partial DNA including the site of polymorphism by using these labeled primers and the antisense primers that specifically hybridize the antisense chain, and which detects polymorphism by measuring the labeling amount of each fluorescent substance in the product of amplification obtained. Furthermore, labeling of the antisense primer herein with biotin, for example, allows the separation of the product of amplification by utilizing the specific binding between biotin and avidin.

Radioactive isotopes, e.g., ³²P, and fluorescent substances, e.g., fluorescein isocyanate, tetramethylrhodamine isothiocyanate, and Texas red, can be exemplified as labeling substances to be used in labeling nucleic acids for polymorphism analysis. The 5′ terminal labeling method using alkali phosphatase and T4 polynucleotide kinase, the 3′ terminal labeling method using T4 DNA polymerase and Klenow fragment, nicktranslation method, random primer method (Molecular Cloning, Third Edition, Chapter 9, Cold Spring Harbor Laboratory Press, New York), and others can be exemplified as labeling methods.

The above-mentioned nucleic acids for polymorphism analysis can be used also under a condition fixed to an insoluble support. Processing of an insoluble support to be used for the fixation to several forms such as chips and beads allows the more simplified analysis of polymorphism by using these fixed nucleic acids.

A nucleic acid sample can be prepared from blood, skin cells, mucous cells, hair, and others from the subject according to known extraction methods, purification methods, and others. In the case of including the gene which is subject to the analysis of polymorphism, the genome DNA of arbitrary length can be used as a nucleic acid sample. Furthermore, it is not necessarily essential to use a nucleic acid sample in which all genes subject to the analysis are present on one nucleic acid. As a nucleic acid sample in the present invention, namely, both material in which all genes subject to the analysis are present on one nucleic acid and material in which genes subject to the analysis are present separately on multiple nucleic acids can be used. Furthermore, material in a fragmented or partial condition may be accepted as long as the site of polymorphism to be analyzed is at least present, although genes subject to the analysis in a nucleic acid sample are not in a complete condition (i.e., a condition in which the full length of the gene is present).

In the case of detecting the type of gene by utilizing the results of the analysis on the above polymorphism of the BMP-4 gene and of the analysis on the above polymorphism of the CTR gene (or the case of diagnosing a genetic risk for bone loss), the analysis of BMP-4 gene polymorphism and the analysis of CTR gene polymorphism shall be conducted separately or simultaneously. In the former case, for example, a nucleic acid sample obtained from the subject is divided into two portions (respective nucleic acid sample may be collected in advance for each analysis) in order to separately analyze each polymorphism. In the latter case, for example, polymorphism can be analyzed by means of the DNA chip or microarray in which the nucleic acid probe which allows the simultaneous detection of each polymorphism is fixed. Furthermore, “simultaneousness” herein does not only imply that all operations of the analysis process are conducted simultaneously but also include the case in which part of operations (e.g., operation to amplify nucleic acid, as well as hybridization or detection of the probe are conducted simultaneously).

Polymorphism of each gene can be analyzed by utilizing mRNA which is the product of transcription of the gene which is subject to the analysis. After extracting and purifying mRNA of the gene which is subject to the analysis from blood, urine, and others of the subject, for example, polymorphism can be analyzed with mRNA as the starting material by conducting methods, e.g., Northern blotting method (Molecular Cloning, Third Edition, 7.42, Cold Spring Harbor Laboratory Press, New York), dot blotting method (Molecular Cloning, Third Edition, 7.46, Cold Spring Harbor Laboratory Press, New York), RT-PCR (Molecular Cloning, Third Edition, 8.46, Cold Spring Harbor Laboratory Press, New York), and methods using the DNA chip (DNA array).

Herein, both the above polymorphism of the BMP-4 gene and the above polymorphism of the CTR gene involve changes in amino acids. Therefore, polymorphism can be analyzed by using the product of expression of each gene. In this case, material, even being partial protein or partial peptide, can be used as a sample for analysis as long as it contains amino acids which correspond to the site of polymorphism.

As methods to analyze these products of gene expression, a method to directly analyze amino acids at the site of polymorphism, a method to immunologically analyze amino acids by utilizing changes in tertiary structure, and others can be mentioned. As the former, a well-known amino acid sequence analysis method (a method utilizing Edman method) can be used. As the latter, ELISA (enzyme-linked immunosorbent assay) using the monoclonal antibody or polyclonal antibody which has an ability to bind specifically to the product of expression of the gene which has either genotype that constitutes polymorphism, radioimmunoassay, immunoprecipitation method, immunodiffusion method, and others can be used.

Information about polymorphism to be obtained by conducting the detection methods in the present invention which are explained above can be utilized to diagnose a genetic risk for bone loss. Namely, the present invention provides methods to diagnose a genetic risk for bone loss after implant treatment, which comprises step for determining the type of gene in a nucleic acid sample based on information about polymorphism that was obtained by the above detection methods (a step for determining the type of gene) and of a step for assessing a genetic risk for bone loss based on the type of gene determined (a step for assessing a genetic risk).

One embodiment in the methods to diagnose a genetic risk in the present invention assesses a genetic risk of the subject (supply source in a nucleic acid sample) by using the genotype determined as a marker after determining the type of gene in a nucleic acid sample (type of gene with respect to the BMP-4 gene) by using the results of the analysis on the above site of BMP-4 gene polymorphism. The results of study conducted by inventors in the present invention revealed that persons with allele A with respect to the BMP-4 gene are prone to develop bone loss. Therefore, the presence or absence of this allele A becomes a marker (risk marker). For example, determination of the presence of allele A in the test sample (detection of allele A) may allow the assessment that a genetic risk for bone loss of the subject is high (high risk) in the process of genetic risk assessment. In the case of determining the genotype (combination of alleles) in the process to determine the type of gene, on the other hand, for example, the genotype determined, AV type or AA type, may allow the assessment that a genetic risk for bone loss of the subject is high (high risk) in the process of genetic risk assessment (especially, the AA type allows the assessment that a genetic risk is higher).

Another embodiment of the present invention use the results of the analysis on the above site of GTR gene polymorphism and the results of the analysis on the above site of BMP-4 gene polymorphism. Namely, the results of these analyses are first used to determine the type of gene in a nucleic acid sample with respect to both the CTR gene and the BMP-4 gene. Next, the combination of the genotypes determined is used as a marker to assess a genetic risk for bone loss of the subject. Utilization of the results of the CTR gene polymorphism analysis and of the results of the BMP-4 gene polymorphism analysis allows the diagnosis of a genetic risk for bone loss at a higher probability of prognosis.

The results of study conducted by inventors in the present invention disclosed that persons with allele T with respect to the CTR gene are prone to develop bone loss. In consideration of this finding and the above similar finding which is associated with polymorphism of the BMP-4 gene, the detection of allele T related to the CTR gene or of allele A related to the BMP-4 gene in the test sample may allow the assessment that the subject's genetic risk for bone loss is high in the process of risk assessment, for example. Furthermore, the detection of both allele T related to the CTR gene and allele A related to the BMP-4 gene in the test sample may allow the assessment that the subject's genetic risk for bone loss is especially high in the process of risk assessment. In the case of determining the genotype (combination of alleles) in the step for determining the type of gene, on the other hand, the genotype of TT type or TC type with respect to the CTR gene in a nucleic acid sample (no regard may be given to the genotype with respect to the BMP-4 gene) or the genotype of AV type of AA type with respect to the BMP-4 gene in a nucleic acid sample (no regard may be given to the genotype with respect to the CTR gene) allows the assessment that the subject's genetic risk for bone loss is high, for example. Concretely, “genotype of the CTR gene/genotype of the BMP-4 gene”, an expression which collectively describes the genotype of the CTR gene and the genotype of the BMP-4 gene in a nucleic acid sample, allows the assessment that the subject's genetic risk for bone loss is high when the genotype in a nucleic acid sample is either of the TC type/AV type, TC type/AA type, TT type/AV type, TT type/AA type, CC type/AV type, CC type/AA type, TC type/VV type, and TT type/VV type. Conversely, the subject's genetic risk for bone loss may be considered low (low risk) when the genotype in a nucleic acid sample is the CC type/VV type.

Furthermore, the subject's genetic risk for bone loss can be considered especially high when the genotype with respect to the CTR gene of the subject is TC type or TT type and the genotype with respect to the BMP-4 gene is concurrently the AV type or AA type (concretely, the case in which the genotype in a nucleic acid sample as expressed in accordance with the above rule is either of the TC type/AV type, TC type/AA type, TT type/AV type, and TT type/AA type).

Diagnosis of a genetic risk for bone loss allows prediction the degree of risk of provoking bone loss (especially early bone loss) around the implant when conducting implant treatment. In other words, the utilization of the diagnostic methods in the present invention allows the assessment of a genetic risk for bone loss around the implant. Awareness in advance of a genetic risk for bone loss securely permits surgery for patients at low risk and, on the other hand, allows the use of prostheses for a long period as possible through countermeasures for patients at high risk, e.g., placement of the long implant as possible and an increase in the number of implants to be placed. In the case of necessary implant treatment despite a high genetic risk for bone loss, furthermore, awareness in advance of the genetic risk allows the minimization of adverse effects due to bone loss through the careful observation of the clinical course after surgery and by quickly instituting appropriate measures when finding bone loss. Thus, a successful evaluation in advance of a genetic risk for bone loss is of high clinical significance.

The second aspect of the present invention provides kits to be used in the above detecting or diagnostic methods in the present invention (kits for detecting the type of gene or kits for diagnosing a genetic risk for bone loss). Such kits contain nucleic acids for analyzing polymorphism BMP-4 (9215T→C) (nucleic acid for polymorphism analysis). As another embodiment, kits are structured which contains nucleic acid for analyzing polymorphism CTR (1377T→C) (nucleic acid for polymorphism) and nucleic acid for analyzing polymorphism BMP-4 (9215T→C) (nucleic acid for polymorphism analysis).

In the analytic methods by which it is applied (e.g., a method which utilizes PCR using above allele-specific nucleic acids and others, PCR-RFLP method, PCR-SSCP method, TaqMan-PCR method, and Invader method), nucleic acids for polymorphism analysis are designed as materials which can specifically amplifies (primer) or specifically detect (probe) the DNA region containing the analysis-subject polymorphism portion or mRNA which corresponds to the region. Concrete examples of kits to be afforded in the present invention are described below.

Kits for detecting the type of gene (or for diagnosing a genetic risk for bone loss), comprising the following nucleic acids: a nucleic acid with a sequence which is complementary to the following region of partial DNA containing the base at position 9215 of the BMP-4 gene whose base at position 9215 is T, or a nucleic acid with a sequence which is complementary to the region of partial DNA containing the base at position 9215 of the BMP-4 gene whose base at position 9215 is C.

Kits for detecting the type of gene (or for diagnosing a genetic risk for bone loss), comprising nucleic acids in the following (1) and (2):
(1) a nucleic acid with a sequence which is complementary to the region of partial DNA containing the base at position 1377 of the CTR gene whose base at position 1377 is T, or a nucleic acid with a sequence which is complementary to the region of partial DNA containing the base at position 1377 of the CTR gene whose base at position 1377 is C.
(2) a nucleic acid with a sequence which is complementary to the region of partial DNA containing the base at position 9215 of the BMP-4 gene whose base at position 9215 is T, or nucleic acid with a sequence which is complementary to the region of partial DNA containing the base at position 9215 of the BMP-4 gene whose base at position 9215 is C.

Kits for detecting the type of gene (or for diagnosing a genetic risk for bone loss), comprising the following set of nucleic acids:
a set of nucleic acids which is designed to specifically amplify the region of partial DNA containing the base at position 9215 of the corresponding BMP-4 gene only in the case that the base at position 9215 of the BMP-4 gene in a nucleic acid sample is T, or a set of nucleic acids which is designed to specifically amplify the region of partial DNA containing the base at position 9215 of the corresponding BMP-4 gene only in the case that the base at position 9215 of the BMP-4 gene in a nucleic acid sample is C.

Kits for detecting the type of gene (of for diagnosing a genetic risk for bone loss), comprising the following sets of nucleic acids (1) and (2):
(1) a set of nucleic acids which is designed to specifically amplify the region of partial DNA containing the base at position 1377 of the corresponding CTR gene only in the case that the base at position 1377 of the CTR gene in a nucleic acid sample is T, or a set of nucleic acids which is designed to specifically amplify the region of partial DNA containing the base at position 1377 of the corresponding CTR gene only in the case that the base at position 1377 of the CTR gene in a nucleic acid sample is C.
(2) a set of nucleic acids which is designed to specifically amplify the region of partial DNA containing the base at position 9215 of the corresponding BMP-4 gene only in the case that the base at position 9215 of the BMP-4 gene in a nucleic acid sample is T, or a set of nucleic acids which is designed to specifically amplify the region of partial DNA containing the base at position 9215 of the corresponding BMP-4 gene only in the case that the base at position 9215 of the BMP-4 gene in a nucleic acid sample is C.

Kits for detecting the type of gene (or for diagnosing a genetic risk for bone loss), comprising the following set of nucleic acids:
a set of nucleic acids which is designed to specifically amplify the region of partial DNA containing the base at position 9215 of the BMP-4 gene and which consists of the sense primer that specifically hybridizes the region of partial DNA containing the base at position 9215 of the BMP-4 gene whose base at position 9215 is T and/or the sense primer that specifically hybridizes the region of partial DNA containing the base at position 9215 in the BMP-4 gene whose gene at position 9215 is C and of the antisense primer that specifically hybridizes a partial portion of the BMP-4 gene.

Kits for detecting the type of gene (or for diagnosing a genetic risk for bone loss), comprising the following sets of nucleic acids (1) and (2):
(1) a set of nucleic acids which is designed to specifically amplify the region of partial DNA containing the base at position 1377 of the CTR gene and which consists of the antisense primer that specifically hybridizes the region of partial DNA containing the base at position 1377 in the CTR gene whose base at position 1377 is T and/or the antisense primer that specifically hybridizes the region of partial DNA containing the base at position 1377 in the CTR gene whose base at position 1377 is C and of the sense primer that specifically hybridizes a partial region of the CTR gene.
(2) a set of nucleic acids which is designed to specifically amplify the region of partial DNA containing the base at position 9215 of the BMP-4 gene and which consists of the sense primer that specifically hybridizes the region of partial DNA containing the base at position 9215 in the BMP-4 gene whose base at position 9215 is T and/or the sense primer that specifically hybridizes the region of partial DNA containing the base at position 9215 in the BMP-4 gene whose base at position 9215 is C and of the antisense primer that specifically hybridizes a partial region of the BMP-4 gene.

Kits for detecting the type of gene (or for diagnosing a genetic risk for bone loss), comprising the following nucleic acids:
a set of nucleic acids which consists of the first nucleic acid that specifically hybridizes a partial region containing the base which corresponds to the base at position 9215 in the antisense chain of the BMP-4 gene whose base at position 9215 is T and that is labeled with the first labeling substance, of the second nucleic acid that specifically hybridizes a partial region containing the base which corresponds to the base at position 9215 in the antisense chain of the BMP-4 gene whose base at position 9215 is C and that is labeled with the second labeling substance, and of the third nucleic acid that specifically hybridizes a partial region of the sense chain of the BMP-4 gene and that can specifically amplify the region of partial DNA containing the base at position 9215 of the BMP-4 gene in concurrent use with the above first or second nucleic acid.

Kits for detecting the type of gene (or for diagnosing a genetic risk for bone loss), comprising the following sets of nucleic acids (1) and (2):
(1) a set of nucleic acids which consists of the first nucleic acid that specifically hybridizes a partial region containing the base at position 1377 in the sense chain of the CTR gene whose base at position 1377 is T and that is labeled with the first labeling substance, of the second nucleic acid that specifically hybridizes a partial region containing the base at position 1377 in the sense chain of the CTR gene whose base at position −850 is C and that is labeled with the second labeling substance, and of the third nucleic acid that specifically hybridizes a partial region of the antisense chain of the CTR gene and that can specifically amplify the region of partial DNA containing the base at position 1377 of the CTR gene in concurrent use with the above first or second nucleic acid.
(2) a set of nucleic acids which consists of the first nucleic acid that specifically hybridizes a partial region containing the base which corresponds to the base at position 9215 in the antisense chain of the BMP-4 gene whose base at position 9215 is T and that is labeled with the first labeling substance, of the second nucleic acid that specifically hybridizes a partial region containing the base which corresponds to the base at position 9215 in the antisense chain of the BMP-4 gene whose base at position 9215 is C and that is labeled with the second labeling substance, and of the third nucleic acid that specifically hybridizes a partial region of the sense chain of the BMP-4 gene and that can specifically amplify the region of partial DNA containing the base at position 9215 of the BMP-4 gene in concurrent use with the above first or second nucleic acid.

In each of the above kits, materials, e.g., not less than one regents (e.g., buffer, reagent for reaction, and reagent for detection) may be combined in response to the usage of the kit.

The present invention is hereafter explained in more detail by using Examples.

### EXAMPLE 1

<Study of the Relationship Between BMP-4 Gene Polymorphism and Early Bone Loss which is Observed Around the Bone Implant>

1. Materials and Methods

1-1. Selection of Subjects

Subjects were 41 healthy Japanese people without consanguinity who underwent implant treatment of the maxilla or mandible between 1999 and 2001 (16 males and 25 females); ages ranged between 29 and 74 years old (mean: 54.8±9.4 years old). Medical history, smoking history, and presence or absence of menstruation were recorded, and ossein was evaluated in compliance with the classification of Leckholm and Zarb (U. Lekholm and G. A. Zarb. 1985. Patient selection and preparation. In: Branemark P-I, Zarb G A, Albrektsson T (eds). Tissue-Integrated Prostheses: Osseointegration in Clinical Dentistry. Chicago: Quintessence: 199-209). Furthermore, the distribution of BMP-4 genotype polymorphism was examined by considering 50 healthy Japanese people without consanguinity who were not affected with oral disorder (23 males and 27 females; ages: 30-70 years old, mean: 50.9±11.9 years old) as the control group. Patients, into whom one oral and maxillofacial surgeon placed a blast-processed implant (Astra Tech, Molndal, Sweden) and who showed bone loss around the implant at the time of second surgery, were considered to constitute the control group.

1-2. Surgical Procedures

All implants were placed according to the usual 2-step surgical procedure under clean environments. Second surgery was conducted after an appropriate period of healing (mandible: 4.5 months in average; maxilla: 6.9 months in average), and one observer who was completely unaware of information about the patient evaluated the bone level in an open manner.

1-3. Extraction of DNA and Analysis of Genotype

Polymorphism within the exon 4 of the BMP-4 gene (14q21, exon 4, 9215→C, refSNP ID: rs17563 (http://www.ncbi.nlm.nih.gov/SNP/snp_ref.cgi?rs=17563), Shore E M, Xu M, Shah P B, et al. The human bone morphogenetic protein 4 (BMP-4) gene: Molecular structure and transcriptional regulation. Calcif Tissue Int 1998;63:221-229) was subject to the analysis.

DNA of the patient was extracted from the peripheral blood collected by using a DNA extraction kit (Qiagen Inc., Valencia, Calif., USA). Polymorphism of the BMP-4 gene was determined by the polymerase chain reaction-restriction fragment length polymorphism (PCR-RFLP) method. The base sequences of the primer which was used for amplifying DNA containing the site of polymorphism in BMP-4 gene polymorphism are as follows:

| | |
|---|---|
| Forward: | |
| 5′-GCTATCTCTTGACTCTTCCATC-3′ | (SEQ ID NO: 3) |
| Reverse: | |
| 5′-CATAGTTTGGCTGCTTCTCC-3′ | (SEQ ID NO: 4) |

Following the 3-hour digestion of 2 μL of a PCR product with Hphl, 3% agarose was used to separate DNA by electrophoresis. Agarose gel was subjected to ethidium bromide staining in order to detect DNA under ultraviolet ray. According to the length of its fragment peptide, the genotype which provided fragment peptides 172 bp and 232b p was considered to be allele V, and the genotype which provided only a fragment peptide 404 bp to be allele A.

1-4. Statistical Analysis

Chi-square test and Fisher's exact probability test were used to statistically analyze gender, smoking history, presence or absence of menstruation, ossein, and distribution of BMP-4 gene polymorphism in the bone loss group and the bone nonloss group. Furthermore, Student's t-test was used to test age in the bone loss group and the bone nonloss group. In addition, logistic analysis was made which considered known risk factors for bone loss, e.g. smoking history, menopause, and ossein. Odds ratio and 95% confidence interval for the BMP-4 genotype were calculated. In all the tests, level of significance was set to less than 0.05.

2. Results

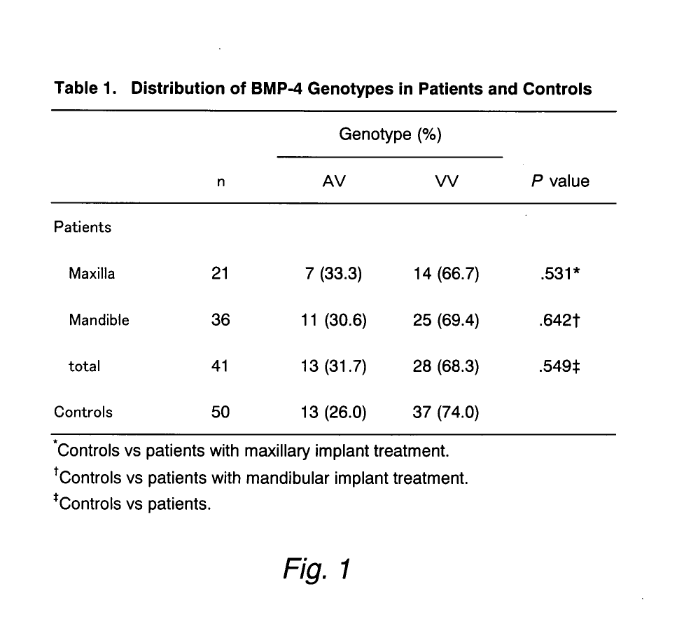

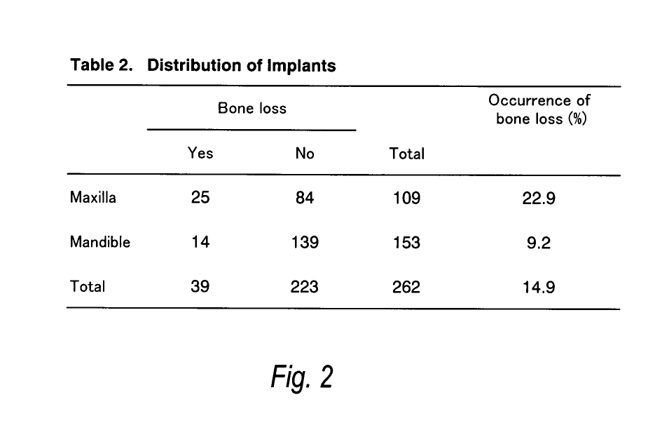

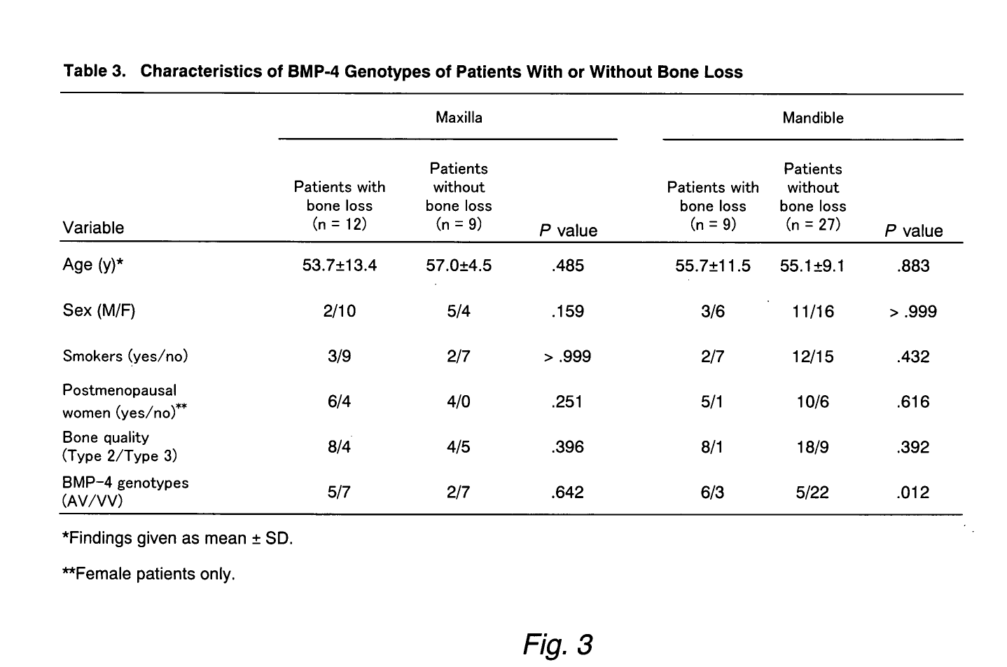

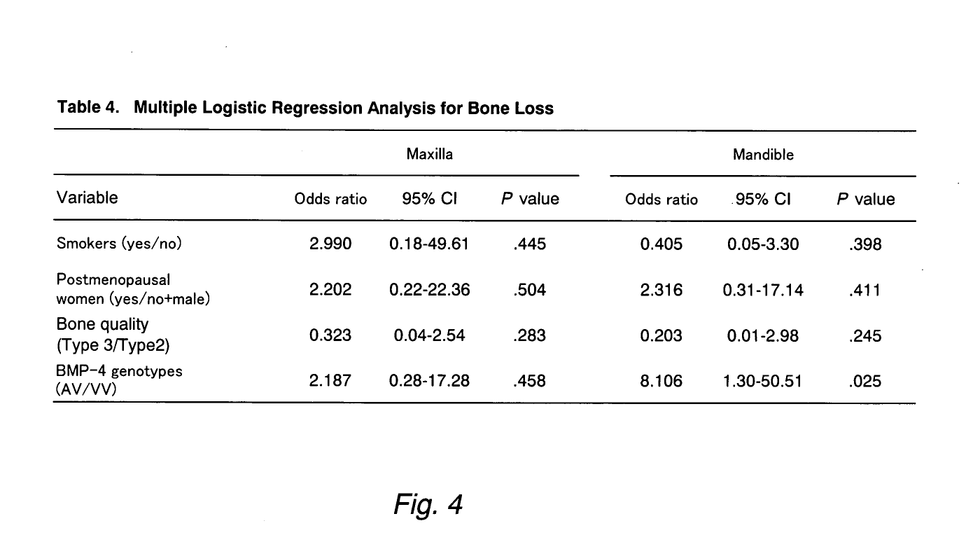

No significant difference was found between the implant-placed patient group and the control group with respect to the distribution of BMP-4 gene polymorphisms, i.e., AV type and VV type [FIG. 1 (Table 1)]. A total of 262 implants, i.e., 109 in the maxilla and 153 in the mandible, were implanted. Consequently, early bone loss was observed in 25 implants (22.9%) in the maxilla and in 14 implants (9.2%) in the mandible [FIG. 2 (Table 2)]. In the mandible, the distribution of BMP-4 gene polymorphisms (AV type and VV type) showed a significant difference (P=0.012) between the bone loss group and the bone nonloss group. In the maxilla, however, no significant difference was found. No significant difference was found between the bone loss group and the bone nonloss group with respect to age, gender, smoking history, presence or absence of menstruation, and distribution of ossein [FIG. 3. (Table 3)]. Furthermore, logistic analysis of bone loss in the mandible revealed a significant difference (P=0.025; FIG. 4 (Table 4)] as manifested by an odds ratio of 8.106 in the AV type against the VV type of the BMP-4 gene.

3. Discussion

No significant difference was found between the implant-placed patient group and the control group with respect to the distribution of BMP-4 gene polymorphisms (AV type and VV type), which led us to consider that subjects were not specific genetically and constituted a population which was suitable as the research subject. The following have been reported as risk factors for bone loss around the implant: smoking history (C. A. Bain and P. K. Moy. 1993. The association between the failure of dental implants and cigarette smoking. Int J Oral Maxiollofac Implants 8,6:609-615; L. W. Lindquist, G. E. Carlsson and T. Jemt, 1996. A prospective 15-year follow-up study of mandibular fixed prostheses supported by osseointegrated implants. Clinical results and marginal bone loss. Clin Oral Implants Res 7,4:329-336); ossein (S. R. Bryant. 1988. The effects of age, jaw site, and bone condition on oral implant outcomes. Int J Prosthodont 11,5:470-490; R. A. Jaffin and C. L. Berman. 1991. The excessive loss of Branemark fixtures in type IV bone; a 5-year analysis. J Periodontol 62,1:2-4); and osteoporosis (Linder L, Carlsson A, Marsal L, Bjursten L M, Branemark P-I. Clinical aspects of osseointegration in joint replacement. A histological study of titanium implants. J Bone Joint Sug [BR], 1988;70:550-555), etc. However, these risk factors showed no significant difference with respect to early bone loss in the present study. Furthermore, implants which were placed in the present study showed no complications at all, e.g., infections and exposure of the cover screw, which led us to consider that early bone loss was provoked not by known risk factors but by possible involvement of the individual's constitution.

FIG. 3 indicated that patients with the AV type of the BMP-4 gene are more prone to develop early bone loss in the mandible than patients with the VV type. Furthermore, logistic analysis also provided a similar result (FIG. 4). However, the 95% confidence interval was as broad as 1.30-50.51 because the number of subjects in the present study was small. After implant placement, bone formation occurs around the implant. Remodeling is considered to occur during the period of healing, and BMP-4 is regarded as one of proteins which are involved in remodeling. The target of the present study is polymorphism which is observed within the BMP-4 gene exon, and replacement of an amino acid (Val→Ala) occurs. Therefore, the possibility that gene polymorphism modifies the phenotype of the BMP-4 gene, decreases function or reduces the content of the BMP-4 gene in the AV type compared to the VV type, and provokes bone loss around the implant.

The present study led us to consider that polymorphism of the BMP-4 gene is one of risk factors for early bone loss around the implant. The development of early bone loss accelerates bone loss after a prosthetic approach and possibly and eventually leads to the failure of implant treatment. To preoperatively comprehend a genetic risk for bone loss is considered to contribute to the improvement of the implant treatment success rate. Therefore, the genetic demonstration of risk factors which are related to early bone loss was considered to lead to a new strategy for improving the success rate.

### EXAMPLE 2

<Study of the Relationship Between Calcitonin Receptor Gene Polymorphism/Bone Morphogenetic Protein-4 Gene Polymorphism and Early Bone Loss Around the Bone Implant>

1. Materials and Methods

1-1. Selection of Subjects

Thirty-six Japanese patients, who underwent implant treatment for mandibular teeth defect by the same surgeon at the same dental clinic between 1999 and 2001 [14 males and 22 females; ages ranged between 29 and 74 years old (mean: 55.3 years old; the treatment group was identical to the group in Example 1], was considered to constitute the implant treatment group. Of patients in the implant treatment group, furthermore, those who developed early bone loss around the implant were considered to constitute the bone loss group, and those who showed no bone loss the bone nonloss group. At the time of treatment, systemic or dental history, smoking history, and presence or absence of menstruation were recorded. Furthermore, ossein was evaluated in accordance with the classification of Leckholm and Zarb (U. Lekholm and G. A. Zarb. 1985. Patient selection and preparation. In: Branemark P-I, Zarb G A, Albrektsson T (eds). Tissue-Integrated Prostheses: Osseointegration in Clinical Dentistry. Chicago: Quintessence: 199-209). On the other hand, 50 healthy Japanese people without systemic disorder and dental disorder (23 males and 27 females; ages: 30-70 years old, mean: 50.9 years old) were considered to constitute the control group. Prior to treatment, the signs of consent and approval were obtained after having provided written and oral explanations about the objectives and contents of research to all the subjects.

1-2. Surgical Procedures

The implant was placed under local anesthesia and clean environments. Following the incision of the alveolar crest, the mucoperiosteal flap was prepared buccolingually and the implant was placed up to a level at which the rough surface was buried within the bone according to the usual 2-step method. The mucoperiosteal flap was restored to the position after putting a cover screw. The suture was removed 10 days after vertical mattress suture. To prevent postoperative infections, 2 g of fosfomycin and 600 mg of clindamycin were administered by drip infusion during surgery, and cefditoren pivoxil 300 mg daily was administered orally for one week after surgery. Subsequent to week 2 after surgery, the inner surface of the denture in use was adjusted with a soft backing material not to impede healing around the implant; the denture was used as the temporary denture.

After an appropriate period of healing, soft tissues were elevated to conduct second surgery. The position of the implant was verified to expose the cover screw by the successive incision of the alveolar crest. One observer physician, who was completely unaware of the genotype, smoking history, and ossein of the subject, evaluated the status of bone around the implant. Bone level was determined by measuring with a caliper the height from the top of the rough surface of the implant to a level where bone is in the first contact with the implant.

1-3. Extraction of DNA and Analysis of Genotypes

Polymorphism within the exon 3 of the CTR gene (7q21, 3 region, 1377T→C, refSNP ID: rs 1801197 (http://www.ncbi.nlm.nih.gov/SNP/snp_ref.cgi?rs=1801197) and polymorphism within exon 4 of the BMP-4 gene (14q21, exon 4, 9215T→C, refSNP ID: rs17563 (http://www.ncbi.nlm.nih.gov/SNP/snp_ref.cgi?rs=17563) were subject to the analysis.

DNA of the patient was extracted from the buccal mucosa collected by using a DNA extraction kit (Qiagen Inc., Valencia, Calif., USA). Furthermore, DNA containing the site of polymorphism in CTR and BMP-4 gene polymorphisms was amplified by according to the chain reaction (PCR) method. The base sequences of each primer are as follows:

For CTR:

| | |
|---|---|
| Forward: | |
| 5′-TTCAGTGGAACCAGCGTTGG-3′ | (SEQ ID NO: 5) |
| Reverse: | |
| 5′-CTCAGTGATCACGATACTGT-3′ | (SEQ ID NO: 6) |

For BMP-4:

| | |
|---|---|
| Forward: | |
| 5′-GCTATCTCTTGACTCTTCCATC-3′ | (SEQ ID NO: 3) |
| Reverse: | |
| 5′-CATAGTTTGGCTGCTTCTCC-3′ | (SEQ ID NO: 4) |

Following electrophoresis of the amplified product with agarose gel, agarose gel was subjected to ethidium bromide staining to detect DNA under ultraviolet ray. The type of gene was determined according to the restriction fragment length polymorphism (RFLP) method. After the 3-hour digestion of 3 μL of PCR product with Alul in the CTR gene and with Hphl in the BMP-4 gene, 3% agarose gel was used to separate DNA by electrophoresis. According to the length of its fragment peptide, the genotype of the CTR gene which provided fragment peptides 108 bp and 120 bp was considered to be allele T, and the genotype which provided only a fragment peptide 228 bp to be allele C. Similarly, the genotype which provided fragment peptides 172 bp and 232 bp in the BMP-4 gene was considered to be allele V, and the genotype which provided only a fragment peptide 404 bp only to be allele A.

1-4. Statistical Analysis

Chi-square test was used to test the genotype expression rate in the implant treatment group and the control group. Chi-square test and Fisher's exact probability test were used to statistically analyze gender, smoking history, presence or absence of menstruation, ossein, genotype, and frequency of allele in the bone loss group and the bone nonloss group. Furthermore, Student's t-test was used to test age in the bone loss group and the bone nonloss group. In addition, odds ratio and 95% confidence interval for the genotype and allele were also examined. In all the tests, level of significance was set to less than 0.05.

2. Results

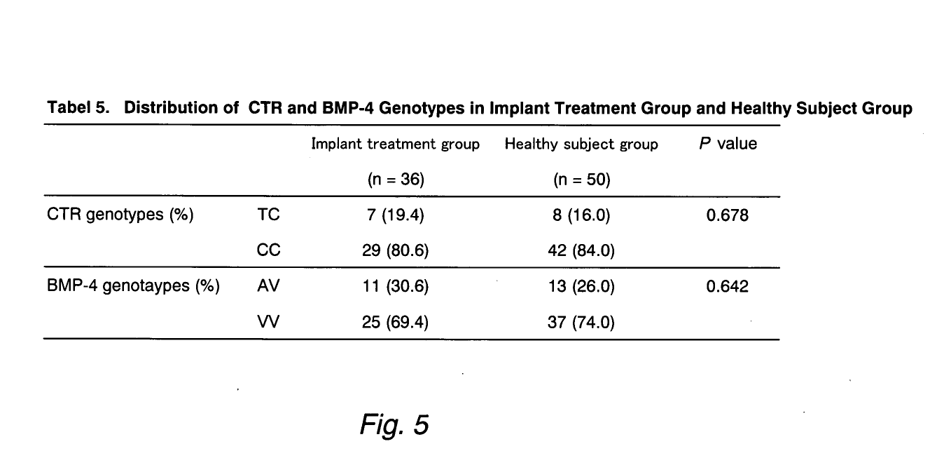

Based on FIG. 5 (Table 5), no significant difference was found between the implant treatment group and the control group with respect to the development of polymorphism of the CTR and BMP-4 genes.

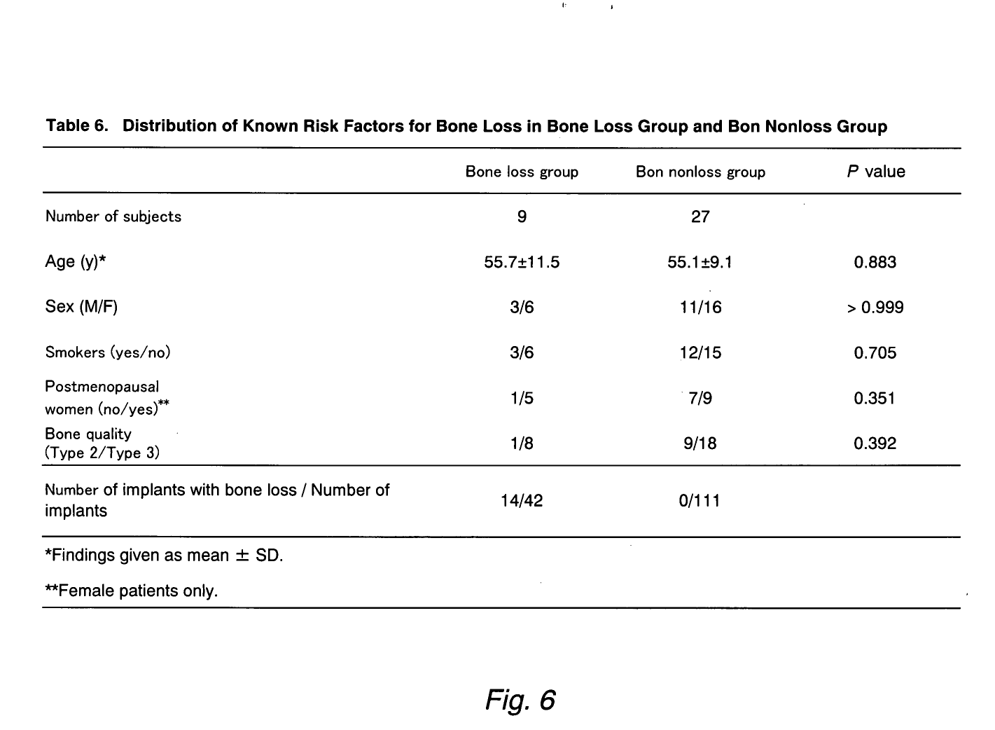

Based on FIG. 6 (Table 6), furthermore, no significant difference was found between the bone loss group and the bone nonloss group with respect to age, gender, smoking history, presence or absence of menstruation, and ossein. One hundred and fifty-three implants were implanted in 36 patients, and early bone loss was observed in 14 implants (mean bone loss: 1.5±0.5 mm). The mean period of healing was 4.1 months (SD: 0.9 month, 2.4-6.0 months). Infection and exposure of the cover screw were not observed in any implants, and osseointegration was obtained clinically and radiologically.

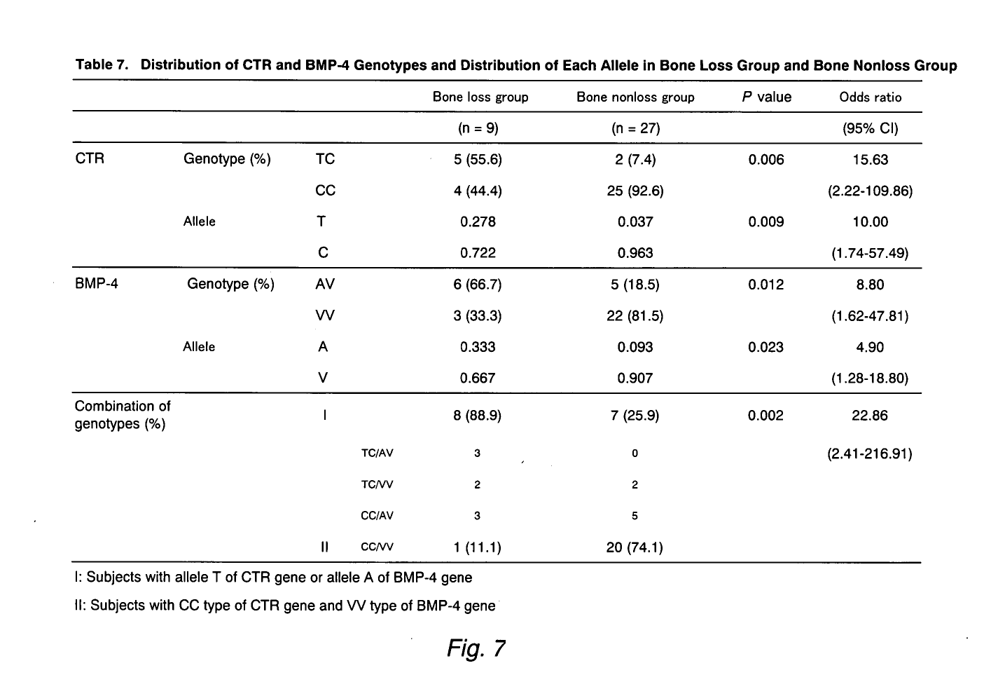

Based on FIG. 7 (Table 7), a significant difference was found between the bone loss group and the bone nonloss group with respect to the genotype of the CTR gene and to the distribution of alleles (odds ratio: 15.63; 95% confidence interval: 2.22-109.86; P=0.006; odds ratio: 10.00; 95% confidence interval: 1.74-57.49; P=0.009). Similarly, a significant difference was found between the bone loss group and the bone nonloss group with respect to the genotype of the BMP-4 gene and to the distribution of alleles (odds ratio: 8.80; 95% confidence interval: 1.62-47.81; P=0.012; odds ratio: 4.90; 95% confidence interval: 1.28-18.80; P=0.023). Furthermore, a significant difference was also found between the bone loss group and the bone nonloss group with respect to the genotype which combined the genotypes of the CTR and BMP-4 genes (odds ratio: 22.86; 95% confidence interval: 2.41-216.91; P=0.002). Of 36 patients in the implant treatment group, none of them was a carrier who had the TT type of the CTR gene and AA type of the BMP-4 gene.

3. Discussion

The present study genetically examined the incidence of early bone loss at the time of second surgery in the implant treatment of the mandible. First, it is necessary to examine whether or not persons, whom the inventors of the present invention considered subjects were appropriate. Therefore, the distribution of genotypes of the CTR and BMP-4 genes was examined in the healthy subject group and the implant treatment group. Based on FIG. 5, no significant difference was found between the healthy subject group and the implant treatment group with respect to the distribution of the TC type and CC type of the CTR gene and to the AV type and VV type of the BMP-4 gene. Therefore, the implant treatment group in the present study was considered not to constitute a genetically specific population, thus leading us to consider that the group was appropriate as the study subject.

Based on FIG. 6, no significant difference was found between the bone loss group and the bone nonloss group with respect to risk factors for systemic bone loss, e.g., ossein (S. R. Bryant. 1988. The effects of age, jaw site, and bone condition on oral implant outcomes. Int J Prosthodont 11,5:470-490; R. A. Jaffin and C. L. Berman. 1991. The excessive loss of Branemark fixtures in type IV bone; a 5-year analysis. J Periodontol 62,1:2-4); smoking history (C. A. Bain and P. K. Moy. 1993. The association between the failure of dental implants and cigarette smoking. Int J Oral Maxiollofac Implants 8,6:609-615; L. W. Lindquist, G. E. Carlsson and T. Jemt, 1996. A prospective 15-year follow-up study of mandibular fixed prostheses supported by osseointegrated implants. Clinical results and marginal bone loss. Clin Oral Implants Res 7,4:329-336), and menopause. Furthermore, infections and exposure of the cover screw were not observed in all the implants placed, which led us to consider that there was no causality between early bone loss which is observed at the time of second surgery and known risk factors for bone loss.

Based on FIG. 7, the TC type and allele T expression rates of the CTR gene were statistically significantly high in the bone loss group compared to the bone nonloss group, with the odds ratios of 15.63 and 10.00, respectively (P=0.006, 0.009). Furthermore, the AV type and allele A expression rates of the BMP-4 gene also were statistically significantly high in the bone loss group compared to the bone nonloss group, with the odds ratios of 8.80 and 4.90, respectively (P=0.012, 0.023). However, the odds ratio of BMP-4 gene polymorphism was low compared to that of CTR gene polymorphism, which led to estimate that CTR gene polymorphism, by itself, has a greater effect on bone loss. Subsequently, combined genes were constituted in consideration of the fact that bone turnover is regulated by a number of proteins. A population with allele T of the CTR gene or with allele A of the BMP-4 gene was categorized into group I, and a population without these alleles, i.e., a population with the CC type of the CTR gene and the W type of the BMP-4 gene, was categorized into group II; the relationship between combined genes and bone loss was thus examined. Among patients in the bone loss group, 88.9% of them belonged to group I, while 74.1% of patients in the bone nonloss group belonged to group II. In group I, the incidence of bone loss was statistically significantly high in the bone loss group, with an odds ratio of 22.86 (P=0.002). Furthermore, three of 36 patients in the group I had both the TC type of the CTR gene and the AV type of the BMP-4 gene; however, all the patients belonged to the bone loss group. This finding suggests that the more one patient has genotype polymorphism which is prone to provoke bone loss, the higher a genetic risk of provoking bone loss is. For those patients with these two genotypes in combination, furthermore, countermeasures, e.g., placement of the long implant as possible and an increase in the number of implants to be placed, would possibly lead to the long-term use of the prosthesis. On the other hand, there were 21 subjects who belonged to group II, 20 of whom belonged to the bone nonloss group. This finding indicates that subjects without allele T of the CTR gene and allele A of the BMP-4 gene are less prone to develop bone loss.

The present study demonstrated that the genetic diagnosis of risk of provoking bone loss prior to the onset of implant treatment is possible. Implant treatment involves a great possibility of progressing to a medical lawsuit. Therefore, scientific comprehension of a therapeutic risk and study of therapeutic strategies are considered to bring a great evangel to clinicians.

### INDUSTRIAL APPLICABILITY

The detection methods of the prevent invention afford information about the genes which are involved in bone loss around the implant after implant treatment. The utilization of this information about polymorphism allows the comprehension in advance of a genetic risk of provoking bone loss (a genetic risk for bone loss) around the implant after implant treatment. The present invention (methods for detecting the type of gene or methods for diagnosing) can be utilized conveniently for an in-advance diagnosis when conducting implant treatment in the field of dentistry. However, subjects to which the methods of the present invention are applicable are not limited to these methods. The present invention can be utilized for the in-advance diagnosis when conducting implant treatment in fields other than dentistry, e.g., femoral head treatment by replacement and maxillofacial treatment with prosthetics.

The present invention is not limited only to the description of the above exemplary embodiments. A variety of modifications, which are within the scopes of the following claims and which are achieved easily by a person skilled in the art, are included in the present invention.

The contents of papers, Kokai publications, patent publications, and others, which are expressed in the present specification, shall be incorporated by reference in their entirety.

1. A method for detecting the type of gene in a nucleic acid sample, comprising the following step:
analyzing polymorphism at the base number position 9215 of the bone morphogenetic protein-4 gene in a nucleic acid sample.

2. A method for detecting the type of gene in a nucleic acid sample, comprising the following steps (a) and (b):
(a) analyzing polymorphism at the base number position 1377 of the calcitonin receptor gene in a nucleic acid sample; and
(b) analyzing polymorphism at the base number position 9215 of the bone morphogenetic protein-4 gene in the nucleic acid sample.

3. A method for diagnosing a genetic risk for bone loss after implant treatment, comprising the following steps (i) to (iii):
(i) analyzing polymorphism at the base number position 9215 of the BMP-4 gene in a nucleic acid sample;
(ii) determining, based on the information about polymorphism which was obtained in the step (i), the type of gene in the nucleic acid sample with respect to the polymorphism of the BMP-4 gene; and
(iii) assessing, based on the type of the gene determined, a genetic risk for bone loss.

4. The method for diagnosing a genetic risk according to claim 3, wherein:
the presence or absence of allele A with respect to the polymorphism of the BMP-4 gene is determined in the step (ii); and
in the step (iii), a genetic risk for bone loss is assessed to be high when determining in the step (ii) that allele A is present, and a genetic risk for bone loss is assessed to be low when determining in the step (ii) that allele A is absent.

5. The method for diagnosing a genetic risk according to claim 3, wherein:
the genotype with respect to the polymorphism of the bone morphogenetic protein-4 gene is determined either of the AV type, AA type, and VV type in the step (ii); and
in the step (iii), a genetic risk for bone loss is assessed to be high when determining in the step (ii) that the genotype is the AV type or AA type, and a genetic risk for bone loss is assessed to be low when determining in the step (ii) that it is the VV type.

6. A method for diagnosing a genetic risk for bone loss after implant treatment, comprising the following steps (I) to (V):
(I) analyzing polymorphism at the base number position 1377 of the calcitonin receptor gene in a nucleic acid sample;
(II) analyzing polymorphism at the base number position 9215 of the bone morphogenetic gene-4 in the nucleic acid sample;
(III) determining, based on the information about polymorphism which was obtained by the step (I), the type of gene in the nucleic acid sample with respect to the polymorphism of the calcitonin receptor gene;
(IV) determining, based on the information about polymorphism which was obtained by the step (II), the type of gene in the nucleic acid sample with respect to the polymorphism of the bone morphogenetic protein-4 gene; and
(V) assessing a genetic risk for bone loss based on the types of the genes which were determined in the steps (III) and (IV).

7. The method for diagnosing a genetic risk according to claim 6, wherein:
the presence or absence of allele T with respect to the polymorphism of the CTR gene is determined in the step (III);
the presence or absence of allele A with respect to the polymorphism of the BMD-4 gene is determined in the step (IV); and
in the step (V), a genetic risk for bone loss is assessed to be high when determining the presence of allele T in the step (III) and/or when determining the presence of allele A in the step (IV), and a genetic risk for bone loss is assessed to be low in other cases.

8. The method for diagnosing a genetic risk according to claim 6, wherein:
the genotype with respect to the polymorphism of the calcitonin receptor gene is determined either of the TC type, TT type, and CC type in the step (III);
the genotype with respect to the polymorphism of the bone morphogenetic protein-4 gene is determined either of the AV type, AA type, and VV type in the step (IV); and
in the step (V), a genetic risk is assessed to be high when [the genotype of the calcitonin receptor gene which was determined in the step (III)/the genotype of the bone morphogenetic protein-4 gene which was determined in the step (IV)] is the TC type/AV type, TC type/AA type, TT type/AV type, TT type/AA type, CC type/AV type, CC type/AA type, TC type/VV type, or TT type/VV type, and a genetic risk is assessed to be low when it is the CC type/VV type.

9. The method for diagnosing a genetic risk according to claim 6, wherein:
the genotype with respect to the polymorphism of the calcium receptor gene is determined either of the TC type, TT type, and CC type in the step (III);
the genotype with respect to the polymorphism of the bone morphogenetic protein-4 is determined either of the AV type, AA type, and VV type in the step (IV); and
in the step (V), a genetic risk is assessed to be high when [the genotype of calcitonin receptor gene which was determined in the step (III)/the genotype of the bone morphogenetic protein-4 which was determined in the step (IV)] is the TC type/AV type, TC type/AA type, TT type/AV type, or TT type/AA type, and a genetic risk is assessed to be low when it is the CC type/AV type, CC type/AA type, TC type/VV type, TT type/VV type, or CC type/VV type.

10. A kit for diagnosing a genetic risk for bone loss after implant treatment, comprising the following nucleic acid:
a nucleic acid for analyzing polymorphism at the base number position 9215 of the bone morphogenetic protein-4 gene.

11. A kit for diagnosing a genetic risk for bone loss after implant treatment, comprising the following (1) and (2):
(1) a nucleic acid for analyzing polymorphism at the base number position 1377 of the calcitonin receptor gene; and
(2) a nucleic acid for analyzing polymorphism at the base number position 9215 of the bone morphogenetic protein-4 gene.

12. Fixing nucleic acids for diagnosing a genetic risk for bone loss after implant treatment, comprising the following nucleic acid which is fixed to an insoluble support:
a nucleic acid for analyzing polymorphism at the base number position 9215 of the bone morphogenetic protein-4 gene.

13. Fixing nucleic acids for diagnosing a genetic risk for bone loss after implant treatment, comprising the following (1) and (2) which are fixed to an insoluble support:
(1) a nucleic acid for analyzing polymorphism at the base number position 1377 of the calcitonin receptor gene;
(2) a nucleic acid for analyzing polymorphism at the base number position 9215 of the bone morphogenetic protein-4 gene.


SEQUENCE LISTING (6 sequences)

SEQ ID NO: 1 - DNA, 11233 bp, Homo sapiens
gaattccttccgtagcttcaccagacacctaattggccaagaaggtttgaagacctgatgtggttcttaattggggatggggaattaagggctactgtatctataggattatcttttcacttgcatagacctatttggtgtgttcagggcatagtgatactataattgccatatttaacagtttataaagttcaagcccagcatattctttgcctgtttaatgatgtcttggtatcagccttttaatggtacttatcagcatagaaaatggaaacaaaataacttttaaaacagtagctctcaagctttagtgtgctcagaatgaccagagaaccttgtgaaatatacagatttctgggtccagatctggggcaggaccaggaagtctgcatttcatctgcacccccaccctactctgaggcttatagtcctgagaacatgctttgaaaaaggctgtcccaagggctcgcagacaggctattgaccagctactctttcttgatgttctccaggaaaaacccaacaaaggaatgcctttcattgagtagtagcagcataggagcaatagttgctcctgaattatggtgggtttcccctcttcatcaatgtgctttaagggtacagtttcatttggtctatctaccatgttctataaaaacatgaaaattcacaggtaagtttgagatacagaaaataactaaactgattcttctcacgaactctgatcactaggctgtggttgatttagctctctaaccaacaagtaatttgttctttggcatgagtaaggggggaaaaggaggagtgggtaaaagcagctgataacagatggcttgcgcccatctaaaatgtggggagagaaataaagctgtcccaagagaactaaagctgagttctctcgtcatatatctgaagattcatatcaggggtctaaacatggtatgtcgggtagcttaattggaaactcctggactgtgagtgtcacagactcatggatgggccaatcagtggccactttagtgtctgggctgcagcaaaatgagacaatagctgtcattcacaaacctttggaattaaaaaaaccccgaaatgacattggtgctttaaagtaaaataaagtcctgcctttaagtccagcatatcactgttgtttctgagtttaaatattaagaaccacatttcgttaatgattaaaacaacagtgattgatttaggggctcagtgagcatttaatctgtcctgacttcaggtaccatgctaaaggagcacaatgcctgatgctgcaggagaaacattaggtaactatttaatggagttttaattttctgttattatttttaataattaattgtgattttgactatttggaagctacaggtatattttgtcctccttttggggtggtgttattgccctgccctgttttaatcagtggttcttagagaaagtgaactcaggagtgacttaaaatgaaggaagacggactttggctaaaattacaattaaataatcaaatcattttcaaatataaagggagcatgcagatgatctggcccaatcctttcattctgcagatgagaaaactgagactcataggaatgaaaagacttgcccaaagccatacagcttgtttctgttgtttggtgcattaggccaaaagacctaggcctaatagatggaaaagatggcaggatgtcttggccttgctctgacagttgcttctctgatctcagatatttcccaccctttgtaatctgtgttccacacaggaagtagttcttgttttttaaatatcgaaggtgtataaacgtaaagtttttatagatgagccacccagggccaatatctgtttaagtaaagacctaaatgctttgcagagacagtaaagtgtcatgtctgtcccagggaaagaaatccaggacaggaaatgctcagtcttccagcactcctctggctacctggagctcaggctatgagcctcaacccctccctgaagcattagctctggagcagaggctgtgatttacttcagagatctgggcaagtccctttaacctggtagtccttcctttccttgtttgtaaaacagagagatgaggctgatagctccctcacagctccatcagaggcagtgtgtgaaattagttcctgtttgggaaggtttaaaagccaccacattccacctccctgctaatatgattactaaaatgtttttatatgaaagggccaattcctcatctcccctcttcctttaaaaacagaccaaggggcatcttttcttgtctccctgtggcctaaaaggttactgcttctgtggttatctccttggaaagacagagtgtcaggactcttaggtacaccaaaaatgaacaaaaaaatcaacaacaaccataacaccaacaaaaataactgctgtgtcggttcttaagacggcttctgagctagaaacagatttttctaactgtaaaaaacgtggccccagcctgtctgcaggccacctctgtctttaggccttggggggaggagggaagtgagctcatttactggggtctacctcagggtcatcaccaaggtgttctacaaaacgcactttaagaatgttttggaaggaaattcaccttttaacagcccaagaggtatctctctctggcacacagttctgcacacagcctgtttctcaacgtttggaaatcttttaacagtttatggaaggccaccttttaaaccgatccaacagctcctttctccataacctgattttagaggtgtttcattatctctaattactcagggtaaatggtgattactcagtgttttaatcatcagtttgggcagcagttacactaaactcagggaagcccagactcccatgggtatttttggaaggtacggcgactagtcggtgcatgctttctagtacctccgcacgtggtccccaggtgagccccagccgcttcccagagctggaggcagcggcgtcccagctccgacggcagctgcggactcgggcgctgcctgggcttccgggacccgggcctgctaggcgaggtcgggcggctggaggggaggatgtgggcggggctcccatccccagaaagggaggcgagcgagggaggagggaaggagggaggggccgccggggaagaggaggaggaaggaaagaaagaaagcgagggagggaaagaggaggaaggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggtgagcaaggctacctggtgaggggagacaggcagagggggtctaggagcctccttggggggaagaagctggtcacaggctgtgaccgaggcaaaaggtggcctaattattttccaatagtggtgctggaggtggggatgctggcgctgaaagacctttaaatatcggctactgcccctgcccaggccttctctgtccagcagtccctgggagattctcacctttgggaagtgcggggcaggagagcagaaacaagagaagcccttggtaggggggtcgttgggaaaaactgtggggtcttgggctgaacgcgttgcccacgggctggaggttgcgatccccggacggaaagcgcgggaggaggaaggagagaaccggctctgaggtccagagagagtgagggggcagagcgacggcgagatggggagagaacacctagctggagcaggttctgcggtagagagcgcagtcctgctggcctctggagagtgcgcgccgctacggaggctgcgtcgaggggagtgtcacccaatctggcccccagctggcggggcgccctgagagcttgcgaactgcagttgcaggacgcgccttctccacgagctattttcgtcgacttgcggaacccaaggaacctcgcctctatcatttcacggtgtagggtccctagagacgacagccaagatcccaggggctcccaggacgcttgttcctgcggtgtcgtgtcctatggggagttcctggcgggacgaaaggcggacgcgcggctcttcctggccctccaggcccggaaccgacgggaaaggttcccgtgattcccgagtccctgcaggcttcttccagcgggagttggtccgggggccttagaggcctccaagcactgctttggaggatggtttccaaggatcgcggtttgtgagttgaaggctttgtgagaggttaaacccccaaaagatacatacttggtaaactgaggctacctgtaaacacatttcggcattaggagaagattcgagtagggaagtgaaggacaaccaccccgagttacattcctttcccccaataaaaagctctggggatgaaagttcttttggcttttatcttttcgatttaaaaatttgagaagaaaaatgtgactagagatgaatcctggtgaatccgaaattgaaacacaactcccccttccccttcctatcctctcggttttagaaccgcgctctcccgccccaggagattccttggggccgagggttttccggggaacccgggcgcccgccccttctactgtccctttgccccgcgggcacagcttgcctccgtctgctttctctacttctggacctctcctcgccgggctttttaaagggcttctgcgtctcaaaacaaaacaaaaaaaccctttgctcttcccaaccctttcgcagcccgccccagcggtggcgcgggaccagcaaaggcgaaagccgcgcggctcttgccgggcgcggacggtcgcgcaggggcgcccgcggcctccgcacccggacctgaggtgttggtcgactccgggcatccacggtcgggagggagggctgagctgttcgatcctttacttttcttcctcaaagtctacctgccaatgcccctaagaagaaaaccaagtatgtgcgtggagagtggggcggcaggcaacccgagttcttgagctccggagcgacccaaagcagcaactgggaacagcctcaggaaagggaggtcgggtggagtgggctttggggcaggagtcatggggcccgggccccggggacgacctggcgctcccggccctgctgaacgctgagttgcgcctagtcgggttttcgaagaggcccttgcgcagagcgacccacgcgcgcggcagcatcttcgattagtcaggacatcccagtaactgcttgaactgtaggtaggtaaaattcttgaaggagtatttgctgcgtgcgactctgctgctggtgcaacggaggaagggggtgggggaaggaagtggcgggggaaggagtgtggtggtggtttaaaaaataagggaagccgaggcgagagagacgcagacgcagaggtcgagcgcaggccgaaagctgttcaccgttttctcgactccggggaacatggtgggatttcctttctgcgccgggtcgggagttgtaaaacctcggccacattaagatctgaaaactgtgatgcgtcctttctgcagagacgcctctttctgaatctgcccggagcttcgagccccggcgtctgtccctcagcctggcatggcttcttcgggggtctgctttgcatggggagaggggccacgcagcggcggactaggtttggggattctcggtaatggacccggagcaatgactaacagccgctccctctcactttcccacagcgatcaccctctaacaccctccctcccattcccggccccgcgcgtgacaaggtcggctgctttcagccgggagctagatcggtggcccggctcttcggagccttagcaggcgttcgccaaggggtgactggctgtcattgggagcaatatttggccttgaggagaccctggggaggaagtggcggggagctcgtgtttgcttgtgtgtgtgtgggggggggggtgtgtgtacacgcgcgtgggcagggtccctctgcgctttcctttttaagtgcctctcggtggtgaggctttgggcgggtgagactttcccgacctcgctcccggccccacttaagccgggttcgagctgggagacgcagtcccttcagtgcgccccaaatcctctggcttcaggtggcccggcgcgggggcccagcacgacgcaccgcgccgagaaccgggttctccgtgcgctgcgccagtagccctgggagcgcggcggccgcggggcaccggccgagggctctgccgagcgccgccgggagctcctcccggaccgctgaggctcgggcggcgggcgcggaggttggcctcgcctggaggggcgggcccgcgaggggcggggggctgtggaggaggggagggcgcgcaggccctttcgccgcctgccgcgggaggggcctcggcgctcacgtgactccgaggggctggaagaaaaacagagcctgtctgcggtggagtctcattatattcaaatattccttttaggagccattccgtagtgccatcccgagcaacgcactgctgcagcttccctgagcctttccagcaagtttgttcaagattggctgtcaagaatcatggactgttattatatgccttgttttctgtcagtgagtagacacctcttccttccccctctccggaattcactctgccctcaccacccctgctcgccggctgtcccttccgtcggacctcctttacaatatccacactctgctccctggcagcactgtcgctcccttcttggcccggcagccggggcgctggaagcgtacgggttccttttaaagtgctgctagcgcgcactcgccctctcagcgttgcaagaaaggggagcgcgagggagctaaagagatgaaagcccggggttgtaccttgagggctaaccactcccttcccctatccaacttgtctgggagagcccccagtgtctccgtggcgcgttcccactctcttgtcaaaactcacagaggtctctccggaatcgtctctcaccccttccctggggatgagcgggcacgatcaggcacttttggctgaatatttcaaactcatcggccacaataaaataagccctcaagccacccggttagctcccagaccaccttctcggcttctggaccctgtcgccctctgtcttcgcccagcccctgcctctcactttccctccctctggctctgaaccaactggaagttgtgaaagttgggctctgagggtggaggaaaagggagagaagctgaaggtctaaagtggagagcaatgccattttaattctccctcccccaccccttttcaccccctcaatgttaactgtttatccttcaagaagccacgctgagatcatggcccagatagcagttaggacaaaaaaagattaacaggatggaggctatctgatttggggttatttgactgtaaacaagttagaccaagtaattacagggcaattcttactttcaggccgtgcatggctgcagctggtgggtgggcgggtggtgtgagggagaagacacaaacttgatctttctgacctgctttccatcttgcccctccatttctagccctaaatgcatatgcagacacatctctatttctccctatttattggtgtttgtttattctttaaccttccactcccctccccctccccagagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaaggtcagtctcttcccccagtctgcgtgggggagggctggtgggactggctagaggggcagtgaaagccctggggaagaagagttcgggttacatcaaaccccagtccaggaggctgaggaacagagctgcttacctccaagaatttgcagagctgccgccgaacttattttttggagacagagggggaggtgttcaggggaaggggaatgacagcactcagacgtgggctagccccagcggtgtgtttttgctatatcaaagccttttctgctaggttttctgcccgtttttttcaaagcacctactgaatttaatattacagctgtgtgtttgtcgggtttattcaataggggccttgtaatccgatctgaatgtttcctagcggatgtttcttttccaaagtaaatctgagttattaatccaccagcatcattactgtgttggaatttattttcccctctgtaacatgatcaacaaggcatgctctgtgtttccaagatcgctggggaaatgtttagtaacatactcaatagtggaagagggagagggtggttgtctccatgtttcctcctgcctgtgctctgttggcccctctttttctttacaaccacttgtaaagaaaactgtggacacaaagccaaggtggggggtttaaaagaggagtctgattgtggtgccatagaggagttgacacatagaaattattagacatatcaaggaggctggatatagtttctgtctttggtgcttgagaaatgctagctacattttgctggtttgttagctgccccacttatctgctccttcaaattaaggggtatgcttattttcccccagtaggtttcccctgcataagcagaattcaccattcattgcccaaccctgagctatctcttgactcttccatctttgaaaaaagttcatatgctttttcttttccccttccttcctaactgtgcctagaacatctggagaacatcccagggaccagtgaaaactctgcttttcgtttcctctttaacctcagcagcatccctgagaacgaggcgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaatggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaatcatgatttttttttctccttaatcctttctcttttccttcgggctcatctcttttgaatgaggcttttttctgttcaggtgagttggaggctggatggaagtcaaaaggtggtacctggaggtggttaagttgtagggacaggaagtaaactgttggcagagagagatggtaattgccagcatgaattgttttctatttctatttaatgttaacaaggatgcagtatcctctcccatctggatgacacatgccttggagaaacactgggatgaaaggagtgtaggtcagattaaagacttcatttcaggccccttgtacatcttctgtttcactcacctgttgaggtgtatcacagctgagcgtgatgaggtctcaaccctagaaaaatgatacccacctctgctttcatgatacctcagggtatctccagttattacaggtaccaatgtgatatttccaaatcaaaactaatttgtacactaacatcataatgtgtgtgtgaaggcatgtttttaaacttattttttttttctccaggtaggactcttttgttttttcttttgtctttttttttttgaaacaagttctctctttgttgccccaggctggtcttgaactcctgggctcaagcaatcttctcatttcggcctctttgggattacaggcatgcactgctattttgtcttttttttttttttgtaacaaataatgtaccctaccttcaaaaagtttgatgactactgttttaatatgccacttgatagaatttcccattgtttcttgactttttcccttgtcctcttttcccaatgtgaaggccttcatcaagtttaggatcccaacagattgggctgggtgggggttgacaatggggtcagatactaaagggtcagaatttctaagcaggcactgtgaaggtgtcccactattatacagaaatctcgag

SEQ ID NO: 2 - DNA, 3331 bp, Homo sapiens
cagaattccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatcttttcattc

SEQ ID NO: 3 - DNA, 22 bp, Artificial
  PCR primer for amplifying a fragment of BMP-4 gene
gctatctcttgactcttccatc

SEQ ID NO: 4 - DNA, 20 bp, Artificial
  PCR primer for amplifying a fragment of BMP-4 gene
catagtttggctgcttctcc

SEQ ID NO: 5 - DNA, 20 bp, Artificial
  PCR primer for amplifying a fragment of calcitonin receptor gene
ttcagtggaaccagcgttgg

SEQ ID NO: 6 - DNA, 20 bp, Artificial
  PCR primer for amplifying a fragment of calcitonin receptor gene
ctcagtgatcacgatactgt